(19) United States
(12) Patent Application Publication
(10) Pub. No.: US 2021/0038697 A1
(43) Pub. Date: Feb. 11, 2021

(54) VIRUS-LIKE NANOCAPSID FOR ORAL DELIVERY OF INSULIN

(71) Applicant: The Regents of the University of California, Oakland, CA (US)

(72) Inventors: R. Holland CHENG, Oakland, CA (US); Chun Chieh Chen, Oakland, CA (US); Mohammad Ali Baikoghli, Oakland, CA (US)

(21) Appl. No.: 16/969,143

(22) PCT Filed: Mar. 13, 2019
(86) PCT No.: PCT/US2019/022137

§ 371 Date: Aug. 11, 2020

Related U.S. Application Data
(60) Provisional application No. 62/642,356, filed on Mar. 13, 2018.

(51) Int. Cl. A61K 38/28; A61K 9/51; A61K 9/00

(52) U.S. Cl. CPC A61K 38/28; A61K 9/5169; A61K 9/0053; C12N 2770/28171; C12N 2770/28123; C12N 2770/28142; C12N 2770/28122

(57) ABSTRACT

Hepatitis E vims (HEV)-based virus like particles (VLP) made with a modified capsid protein containing at least a portion of open reading frame 2 (ORF2) protein and encapsulated insulin protein or insulin encoding nucleic acid are provided. Also provided are methods of targeted delivery of insulin using the HEV VLP.

### RELATED APPLICATION

This application is a 371 U.S. National Stage of PCT/US2019/022137, Internaitonal Filing Date Mar. 13, 2019, which claims priority to U.S. Patent Application No. 62/642,356, filed Mar. 13, 2018, the contents of which are hereby incorporated by reference in the entirety for all purposes.

### STATEMENT AS TO RIGHTS TO INVENTIONS MADE UNDER FEDERALLY SPONSORED RESEARCH AND DEVELOPMENT

This invention was made with government support under contracts AI095382, EB021230, and CA198880 awarded by the National Institutes of Health and the USDA grant of National Institute of Food and Agriculture. The government has certain rights in the invention.

### SEQUENCE LISTING AS A TEXT FILE

The instant application contains a Sequence Listing which has been submitted electronically in ASCII format and is hereby incorporated by reference in its entirety. Said ASCII copy, created on Aug. 7, 2020, is named 81906-1205931_Sequence_Listing.txt and is 62,123 bytes in size.

### BACKGROUND OF THE INVENTION

Virus-like particles (VLPs) can serve as nanocarriers for targeted delivery of diagnostics and therapeutics regimes, such as DNA/RNA and a variety of chemotherapeutics. Hepatitis E virus (HEV) is an enteric-transmitted virus that causes acute liver inflammation in humans. HEV virus-like particles (HEV VLPs) are capsid protein icosahedral cages that can be produced by expression of the major capsid protein HEV Open Reading Frame 2 (ORF2) in a eukaryotic expression system. HEV VLPs are stable in acid and proteolytic environments, a feature that is required for the natural transmission route of HEV. Thus, HEV VLPs represent a promising nano-carrier that can be exploited, e.g., for the delivery of therapeutic agents, imaging agents, or vaccines.

One disease that nona-carriers have been considered for treating is diabetes, a condition that is highly prevalent especially in developed countries. Despite numerous other medicines that have been developed to treat diabetes, insulin remains the first choice to treat type 1 diabetes (T1D) and advanced type 2 diabetes (T2D). Although the morbidity and mortality of diabetes patients has been greatly reduced due to insulin, 60% of the patients still fail to attain long-term glucose control[1]. It is probably caused by the discomfort and stigma connected to the typical usage of needles in insulin administration. On the contrary, oral delivery of insulin is considered convenient, cost effective and the preferred administration method with the highest patient compliance. In addition, the oral route mimics the endogenous insulin secretion pathway from the pancreas to the liver through the hepatic portal vein to achieve better glucose homeostasis[2-4]. The progress of oral insulin delivery has been crippled by the low bioavailability of insulin due to its degradation in the gastrointestinal (GI) tract as protein, and its poor permeability through the intestinal epithelium[4, 5]. Nonetheless, oral delivery is still an attractive alternative over needle injection, especially since the once favorable pulmonary route missed the forecast as a real prospect[6].

Several oral insulin delivery pharmaceutics have proposed utilizing paracellular and/or transcellular transport through the ileum and colon via platforms such as tablets, capsules, intestinal patches, hydrogels, microparticles, and nanoparticles. The status of those oral insulin developments, and the progress in different stages of clinical trials, have been reviewed in several review articles[4, 7-10]. Among them, Oram Pharmaceuticals Inc. in Israel owns patented Protein Oral Delivery (POD') technology, which adopts a three-pronged approach composed of encapsulation, protease inhibitors and a chelating agent. Its clinical trials on both T1D and T2D patients are ongoing. Novo Nordisk A/S in Denmark has conducted Phase 1 and Phase 2 clinical trials with oral insulin tablets based on micromulsions of oil and surfactant or a mixture of fatty-acid derivatives in an enteric-coated gel capsule. Despite its preliminary success in clinical trials, Novo Nordisk made the difficult decision to discontinue its oral insulin development program in the end of 2016 due to the system's low efficiency. Based on the technologies and experiences learned by these pioneer developments, the present inventors seek to address several cost-effective factors such as sufficient bioavailability and the reproducible absorption of insulin, which relates to the understanding of meal-dependent absorption rate and the mass production of oral insulin delivery system.

The development of gene therapy has also been proposed as a possible cure for diabetes since the late 1970s when the insulin gene was cloned and expressed in cultured cells[11]. A Madison, WI-based startup, Insulete, seeks to commercialize a gene therapy that induces insulin production in patient's liver cells. They target the liver, instead of the pancreas, because of its ability to regenerate. In prior animal testing, a single injection of naked insulin DNA plasmid could provide glycemic control for up to six weeks[12]. However, the system lacks specific tissue/cell targeting delivery which still needs to be addressed for an effective treatment. As such, there exists a distinct need for developing new and effective means for insulin delivery in diabetes treatment. The present invention fulfills this and other related needs.

### BRIEF SUMMARY OF THE INVENTION

The present invention provides an HEV VLP intended for targeted delivery of insulin as well as the method of delivery of insulin using such HEV VLP.

In a first aspect, the present invention provides a composition comprising (a) modified capsid protein that comprises at least a portion of hepatitis E virus (HEV) open Reading Frame 2 (ORF2) protein and is able to form an HEV virus like particle (VLP); and (b) insulin either in the form of a protein or a polynucleotide coding sequence encapsulated within the HEV VLP formed by the modified capsid protein. Typically, the modified ORF2 protein is less than full length of the wild-type protein (e.g., any one of those provided in SEQ ID NOs:1-6). The specific modification of the ORF2 protein may be among those described in earlier disclosures by the present inventors, see, e.g., U.S. Pat. Nos. 8,906,862 and 8,906,863, WO2015/179321.

In some embodiments, the modified capsid protein is less than full length of HEV ORF2 protein; it comprises segment 452-606 of the HEV ORF 2 protein of SEQ ID NO:1, 2, 3, 4, 5, or 6; and it comprises a heterologous polypeptide sequence inserted into the portion of HEV ORF2 protein within segment 483-490, 530-535, 554-561, 573-577, 582-593, or 601-603 of SEQ ID NO:1, 2, 3, 4, 5, or 6. In some embodiments, the heterologous polypeptide sequence is inserted immediately after residue Y485 of SEQ ID NO:1, 2, 3, 4, 5, or 6. In some embodiments, the heterologous polypeptide may be involved in targeting liver cells for delivery of insulin, for example, the most widely used homing peptide, RGD (Arg-Gly-Asp) peptide or cyclic RGD peptide[1], which shows strong affinity for integrins vb 3 and vb 5, or homing peptides that specifically target HCC include TTPRDAY (SEQ ID NO: 13) [2], FQHPSFI (SEQ ID NO: 14) (HCBP1) [3], SFSIIHTPILPL (SEQ ID NO: 15) (SP94) [4], RGWCRPLPKGEG (SEQ ID NO: 16) (HC1) [5], AGKGTPSLETTP (SEQ ID NO: 17) (A54) [6], KSLSRHDHIHHH (SEQ ID NO: 18) (HCC79) [7] and AWYPLPP (SEQ ID NO: 19) [8].

In some embodiments, the modified capsid protein is able to form an acid and proteolytically stable HEV VLP and has at least one residue Y485, T489, 5533, N573, or T586 of SEQ ID NO:1, 2, 3, 4, 5, or 6 substituted with a cysteine or lysine, and the cysteine or lysine is optionally chemically derivatized. In some embodiments, the cysteine or lysine is alkylated, acylated, arylated, succinylated, oxidized, or conjugated to a detectable label or liver cell targeting ligand. For example, the detectable label may comprise a fluorophore, a superparamagnetic label, an Mill contrast agent, a positron emitting isotope, or a cluster of elements of group 3 through 18 having an atomic number greater than 20. In some embodiments, the detectable label comprises a gold nanocluster. In another example, the liver cell targeting ligand is the heterologous polypeptide may be involved in targeting liver cells for delivery of insulin, for example, the most widely used homing peptide, RGD (Arg-Gly-Asp) or cyclic RGD peptide[1], or homing peptides that specifically target HCC include TTPRDAY (SEQ ID NO: 13) [2], FQHPSFI (SEQ ID NO: 14) (HCBP1) [3], SFSIIHTPILPL (SEQ ID NO: 15) (SP94) [4], RGWCRPLPKGEG (SEQ ID NO: 16) (HC1) [5], AGKGTPSLETTP (SEQ ID NO: 17) (A54) [6], KSLSRHDHIHHH (SEQ ID NO: 18) (HCC79) [7] and AWYPLPP (SEQ ID NO: 19) [8].

In some embodiments, the composition may further comprises a pharmaceutically acceptable excipient, or it may be formulated for oral administration, for example, for treating diabetes patients.

In a second aspect, the present invention provides a method for targeted delivery of insulin to liver cells, the method including a step of contacting a liver cell with the composition of any variety describe above and herein, especially those with a liver cell targeting ligand such as RGD (cyclic RGD) peptide[1].

In some embodiments, the liver cell is within a patient's body, and the contacting step comprises administration of the composition containing an effective amount of an HEV VLP described above and herein to the patient. In some embodiments, the administration is oral administration. In some embodiments, the modified capsid protein comprises a cysteine or lysine conjugated to a gold nanocluster. In some embodiments, the patient has been diagnosed with diabetes. In some embodiments, the patient is an animal, especially a mammal such as a primate including a human.

### BRIEF DESCRIPTION OF THE DRAWINGS

FIG. 1: The schematic of insulin encapsulated HEVNP. (left panel) The oral delivery pathways of insulin encapsulated HEVNPs. HEVNP will go through GI tract and then to liver via Hepatic portal vein. (right panel)

FIG. 2: TEM micrographs of Insulin (A); Insulin encapsulated HEVNPs (B); The size distribution of insulin encapsulated HEVNPs under TEM observation. Most of them have the size around 52 nm (C); The TEM images of insulin encapsulated HEVNPs. The bar is 100 nm in length.

FIG. 3: TEM micrographs of Insulin encapsulated HEVNPs: without Pepsin treatment as control (A); after (38U/ml) Pepsin treatment at pH3, 37° C. for 5 min (B); after (38U/ml) Pepsin treatment at pH4, 37° C. for 5 min (C). The bar is 100 nm in length.

FIG. 4: Insulin Encapsulation of HEVNP: Optimization of packaging condition to increase the efficiency of insulin encapsulation in HEVNP.

FIG. 5: Insulin Encapsulation of HEVNP: Optimization of packaging condition to increase the efficiency of insulin encapsulation in HEVNP tested by Bradford assay and ELISA; Sonication-mediated payload enhancement (bottom panel).

FIG. 6: Size exclusion column analysis: Shows distinct peaks of insulin and HEVNP with overlapped as shown by ELISA (indicated by the + signs between conditions #16 and #32.

FIG. 7: Insulin Encapsulation of HEVNP: Cryo-electron microscope structure-guided optimization of insulin packaging, followed by 3D modeling of insulin packaging and computational validation of packing mechanism. Electron microscope tomography tilt-series data collection to reconstruct a 3D representation of HEVNP-Insulin.

FIG. 8: High stability and shelf life: The HEVNP-insulin samples were stored in 4C for over one year and examined with cryo-EM. The micrographs show intact particles which shows high stability for storage conditions.

FIG. 9: Enhanced stability of HEVNP via AuNC: CryoArm 300 kV microscopy and 3D image reconstruction of enhanced HEVNP stability via clustered metal atoms based on the capsid surface modulation. High resolution structure determination is the key to optimize HEVNP mucosal delivery.

### DEFINITIONS

As used in this specification and the appended claims, the singular forms “a,” “an,” and “the” include plural reference unless the context clearly dictates otherwise.

“Hepatitis E virus” or “HEV” refers to a virus, virus type, or virus class, which i) causes water-borne, infectious hepatitis; ii) is distinguished from hepatitis A virus (HAV), hepatitis B virus (HBV), hepatitis C virus (HCV), or hepatitis D virus (HDV) in terms of serological characteristics; and iii) contains a genomic region that is homologous to a 1.33 kb cDNA inserted in pTZKF1(ET1.1), a plasmid embodied in a E. coli strain deposited in American Type Culture Collection (ATCC) with accession number 67717.

The terms “capsid protein” and “modified capsid protein,” with reference to HEV, refer to a mature or modified (e.g., truncated, recombinantly mutated, or chemically derivatized) HEV open reading from 2 (ORF2) polypeptide. As used herein, reference to such ORF2 polypeptides or proteins is meant to include the full-length polypeptide, and fragments thereof, and also include any substitutions, deletions, or insertions or other modifications made to the ORF2 proteins. The capsid proteins must be capable of forming a virus like particle (VLP). Typically the capsid protein contains at least residues 112-608 of HEV ORF2, although the capsid protein can tolerate various additional substitutions, deletions, or insertions so long as they are tolerated without abrogating VLP formation.

In one embodiment, the term “modified capsid protein” refers to a capsid protein, or portion thereof (i.e., less than full length of the capsid protein), in which modifications such as one or more of additions, deletions, substitutions are present yet the resultant modified capsid protein remain capable of forming a VLP. These modifications include those described in U.S. Pat. Nos. 8,906,862 and 8,906,863, WO2015/179321. For instance, a heterologous polypeptide may be inserted into the capsid protein or a portion thereof, at locations such as within segment 483-490, 530-535, 554-561, 573-577, 582-593, or 601-603, or immediately after residue Y485, see U.S. Pat. Nos. 8,906,862 and 8,906,863. As an another example, WO2015/179321 describes further examples of modified capsid protein in which a surface variable loop of the P-domain of HEV ORF2 is modified to incorporate one or more cysteines or lysines that are not otherwise present in the wild-type capsid protein sequence. Alternatively, or additionally, the term “modified capsid protein” refers to a capsid protein, or portion thereof, in which the C-terminus (e.g., position 608) of HEV ORF2 is modified to incorporate one or more cysteines or lysines that are not otherwise present in the wild-type capsid protein sequence. Alternatively, or additionally, the term “modified capsid protein” refers to a capsid protein, or portion thereof, in which a cysteine or lysine (e.g., a cysteine or lysine of a surface variable loop of the P-domain of HEV ORF 2 or a cysteine/lysine recombinantly introduced at position 608) is chemically derivatized to covalently conjugate to the protein at least one heterologous atom or molecule. The cysteine or lysine can be inserted such that the HEV ORF2 protein length is increased, or the cysteine or lysine can replace one or more residues of a P-domain surface variable loop and/or C-terminus.

Generally, modified capsid proteins retain the ability to form HEV VLPs. In some cases, the one or more cysteines or lysines are conjugated to a bioactive agent (e.g., a cell-targeting ligand such as the peptide LXY30). P-domain surface variable loops include one or more of, e.g., residues 475-493; residues 502-535; residues 539-569; residues 572-579; and residues 581-595 of HEV ORF 2 (SEQ ID NO:1, 2, 3, 4, 5, or 6). P-domain surface variable loops further include the residues of polypeptides comprising an amino acid sequence that is at least about 80%, 85%, 90%, 95%, 99%, or more identical to one or more of SEQ ID NOS:1, 2, 3, 4, 5, or 6 and that correspond to one or more of residues 475-493; residues 502-535; residues 539-569; residues 572-579; and residues 581-595 of SEQ ID NOS:1, 2, 3, 4, 5, or 6.

As used herein, the term “virus-like particle” (VLP) refers to an icosahedral shell (e.g., T1 or T3) formed by a capsid protein. VLPs are not infectious due to the lack of a viral genome. “VLP” refers to a nonreplicating icosahedral viral shell, derived from hepatitis E virus capsid protein HEV ORF2, a portion thereof. VLPs can form spontaneously upon recombinant expression of the protein in an appropriate expression system. In some embodiments, the VLP is formed from a modified capsid protein, e.g., a capsid protein containing one or more cysteine/lysine residues in a surface variable loop of HEV ORF2, or a portion thereof. An HEV VLP can contain a mixture of modified and/or unmodified HEV ORF2 proteins.

The term “acid and proteolytically stable” in the context of an HEV VLP refers to an HEV VLP that is resistant to the acid and proteolytic environments of a mammalian digestive system. Methods of assessing acid and proteolytic stability are described in Jariyapong et al., 2013, and include, but are not limited to subjecting an HEV VLP to an acid (e.g., pH of, or of about, 6, 5.5, 5, 4.5, 4, 3.5, 3, 2.5, or 2) and/or proteolytic environment (e.g., trypsin and/or pepsin) and examining the contacted HEV VLP by electron microscopy, gel filtration chromatography, or other suitable method to determine whether the quaternary structure (e.g., T=1, T=3, icosahedron, dodecahedron, etc.) of the HEV VLP is retained. A population of HEV VLPs (e.g., modified or unmodified) can be incubated under acid and/or proteolytic conditions for a suitable period of time (e.g., for at least, or for at least about, 1, 2, 3, 4, 5, 10, 15, 20, 30, 45, or 60 minutes) and then tested to determine the extent of quaternary structure retention. In this context, an acid and proteolytically stable modified HEV VLP refers to a modified HEV VLP that when incubated as a population of VLPs under acid and/or proteolytic conditions and assayed by electron microscopy, at least 10%, 25%, 50%, 75%, 90%, 95%, 99%, or 100% of the VLPs of the population retain their quaternary structure.

Alternatively, the HEV VLP can be delivered to a subject via an oral route and the efficiency of delivery assessed by detecting and/or quantifying: (i) an immune response to an antigen within the HEV VLP; (ii) a detectable label conjugated to, recombinantly introduced into, or encapsulated by the HEV VLP; or (iii) a biological response due to delivery to a cell of a bioactive agent associated with (e.g., recombinantly introduced into, conjugated to, or encapsulated by) the HEV VLP. In this context, an acid and proteolytically stable modified HEV VLP refers to a modified REV VLP that retains at least 10%, 25%, 50%, 75%, 90%, 95%, 99%, or 100% of the oral delivery efficacy and/or cell entry activity of an unmodified HEV VLP.

The term “heterologous” as used in the context of describing the relative location of two elements, refers to the two elements such as nucleic acids (e.g., promoter or protein encoding sequence) or proteins (e.g., an REV ORF2 protein, or portion thereof, or modified capsid protein and another protein) that are not naturally found in the same relative positions. Thus, a “heterologous promoter” of a gene refers to a promoter that is not naturally operably linked to that gene. Similarly, a “heterologous polypeptide” or “heterologous nucleic acid” in the context of an HEV VLP or HEV capsid protein is one derived from a non-HEV origin.

Hepatitis E virus (HEV) is known to cause severe acute liver failure. HEV belongs to the genus Hepevirus in the family Hepeviridae. HEV contains a single-stranded positive-sense RNA molecule of approximately 7.2-kb. The RNA is 3′ polyadenylated and includes three open reading frames (ORF). ORF1 encodes viral nonstructural proteins, located in the 5′ half of the genome. ORF2 encodes a protein-forming viral capsid, located at the 3′ terminus of the genome. ORF3 encodes a 13.5-kDa protein, overlapped with C-terminus of ORF1 and N-terminus of ORF2. ORF3 is associated with the membrane as well as with the cytoskeleton fraction.

The term “encapsulation,” or “encapsulated,” as used herein refers to the envelopment of a heterologous substance, such as a heterologous nucleic acid or protein, a chemotherapeutic, an imaging agent, a ferrite nanoparticle etc., within the VLPs defined herein.

The term “bioactive agent” refers to any agent, drug, compound, or mixture thereof that targets a specific biological location (targeting agent) and/or provides some local or systemic physiological or pharmacologic effect that can be demonstrated in vivo or in vitro.

Non-limiting examples include drugs, hormones, vaccines, antibodies, antibody fragments, vitamins and co factors, polysaccharides, carbohydrates, steroids, lipids, fats, proteins, peptides, polypeptides, nucleotides, oligonucleotides, polynucleotides, and nucleic acids (e.g., mRNA, tRNA, snRNA, RNAi, DNA, cDNA, antisense constructs, ribozymes, etc.).

A “pharmaceutically acceptable” or “pharmacologically acceptable” material is one that is not biologically harmful or otherwise undesirable, i.e., the material may be administered to an individual along with the capsid protein or the HEV VLPs or the compositions of the present invention without causing any undesirable biological effects. Neither would the material interact in a deleterious manner with any of the components of the composition in which it is contained.

The term “excipient” refers to any essentially accessory substance that may be present in the finished dosage form of the composition of this invention. For example, the term “excipient” includes vehicles, binders, disintegrants, fillers (diluents), lubricants, glidants (flow enhancers), compression aids, colors, sweeteners, preservatives, suspending/dispersing agents, film formers/coatings, flavors and printing inks.

The term “adjuvant” refers to a compound that, when administered in conjunction with an antigen, augments the immune response to the antigen, but does not generate an immune response to the antigen when administered alone. Adjuvants can augment an immune response by several mechanism including lymphocyte recruitment, stimulation of B and /or T cells, and stimulation of macrophages.

An “immunogenic response” to an antigen or composition is the development in a subject of a humoral and/or a cellular immune response to an antigen present in the composition of interest. For purposes of the present disclosure, a “humoral immune response” refers to an immune response mediated by antibody molecules, while a “cellular immune response” is one mediated by T-lymphocytes and/or other white blood cells. One important aspect of cellular immunity involves an antigen-specific response by cytolytic T-cells (“CTL”s). CTLs have specificity for peptide antigens that are presented in association with proteins encoded by the major histocompatibility complex (MHC) and expressed on the surfaces of cells. CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4+ and CD8+ T-cells. Hence, an immunological response may include one or more of the following effects: the production of antibodies by B-cells; and/or the activation of suppressor T-cells and/or γΔ T-cells directed specifically to an antigen or antigens present in the composition or vaccine of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art.

A “label,” “detectable label,” or “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins that can be made detectable, e.g., by incorporating a radioactive component into the peptide or used to detect antibodies specifically reactive with the peptide. Typically a detectable label is a heterologous moiety attached to a probe or a molecule with defined binding characteristics (e.g., a polypeptide with a known binding specificity or a polynucleotide), so as to allow the presence of the probe/molecule (and therefore its binding target) to be readily detectable. The heterologous nature of the label ensures that it has an origin different from that of the probe or molecule that it labels, such that the probe/molecule attached with the detectable label does not constitute a naturally occurring composition.

The term “treat” or “treating,” as used in this application, describes to an act that leads to the elimination, reduction, alleviation, reversal, or prevention or delay of onset or recurrence of any symptom of a relevant condition. In other words, “treating” a condition encompasses both therapeutic and prophylactic intervention against the condition.

The term “effective amount” as used herein refers to an amount of a given substance that is sufficient in quantity to produce a desired effect. For example, an effective amount of HEV nanoparticle (HEVNP) encapsulating insulin is the amount of said HEVNP to achieve a detectable effect, such that the symptoms, severity, and/or recurrence chance of a target disease (e.g., diabetes) are reduced, reversed, eliminated, prevented, or delayed of the onset in a patient who has been given the HEVNP for therapeutic purposes. An amount adequate to accomplish this is defined as the “therapeutically effective dose.” The dosing range varies with the nature of the therapeutic agent being administered and other factors such as the route of administration and the severity of a patient's condition.

The term “patient” as used herein refers to a vertebrate animal, e.g., of avian or mammalian species, especially a mammal (for example, a bull/cow, pig, sheep/goat, horse, rabbit, rodent, dog, cat, fox, etc.) including a primate such as a chimpanzee, a monkey or a human.

### DETAILED DESCRIPTION OF THE INVENTION

### A. Introduction

This disclosure relates to a viral-based nanocapsid, which is chemically stable and resistant to the enzymatic activities in the gastrointestinal tract, for oral delivery of insulin. As it is well known that certain limitations in the diabetes treatment including poor patient compliance are due to the discomfort and adverse effects associated with the common use of needle injection for insulin administration. Although oral delivery is the most favorable delivery route for insulin, a protein of molecular weight 5.8 kDa, it faces challenges including degradation in the gastrointestinal tract by proteolytic enzymes and severe acid physiological conditions and delivery efficacy following absorption and permeability through the intestinal epithelium. While several systems of oral delivery of insulin have been developed and approved for clinical trials, a number of cost-related factors need to be addressed including the need to improve the low bioavailability, to achieve reproducible absorption, to gain understanding of meal-dependent absorption rate and the mass production of oral administered insulin delivery system.

The Hepatitis E Virus Nanoparticle (HEVNP) is derived from a self-assembling, noninfectious nanocapsids. HEVNP is stable in acidic environment and resistant to proteolytic digestion, thus it possesses a great advantage as an oral delivery vehicle. HEVNP can be orally administered, then transported to the small intestine and ultimately to the liver following HEV's natural transmission route. With its in vitro disassembly/reassembly ability, HEVNP is capable of encapsulating drug or nucleic acids to deliver them through the digestion system in gastrointestinal tract. The specific targeting ligand (e.g., a ligand targeting delivery to the liver) can be linked to the protrusion domain of HEVNP either by genetic engineering or chemical conjugation. The HEVNP structure can be stabilized by conjugating monodispersed gold nano-clusters (AuNCs) for better bioavailability of oral delivered drug (e.g., insulin)[18].

The specific aspects in this disclosure and earlier publications by the present inventors (see, e.g., U.S. Pat. Nos. 8,906,862 and 8,906,863, WO2015/179321) outline HEVNP production as well as methods and applications in surface modification, encapsulation for oral delivery of insulin to liver and mimic its physiological secretion route from pancreas to liver.

The structure stabilized HEVNPs as oral insulin delivery capsule provides the following benefits: (1) eliminating needles, associated risks, and disposal requirements; (2) insulin, either as a polypeptide or a polynucleotide coding sequence itself, can be readily encapsulated into the HEVNP structure in vitro and delivered to liver, even without targeting ligand. However, therapeutic targeting ligand will enable and enhance delivery of insulin (e.g., insulin gene) to pancreas specifically; (3) HEVNP, composed of capsid proteins, can be biodegraded through protein degradation pathway with little toxicological concerns.

The combination of the various versions of insulin encapsulated HEVNPs can be used as combined-modality therapy for better control of blood glucose level in Diabetes patients. A scale-up production and expression of HEVNPs are to be performed following animal tests for cost analysis of the treatment scheme.

### B. Production and Purification of Modified Capsid Proteins and VLP Formation

One aspect of the invention relates to methods for production and purification of capsid proteins and VLPs derived therefrom (See, Expression and self-assembly of empty virus-like particles of hepatitis E virus. Li T C, Yamakawa Y, Suzuki K, Tatsumi M, Razak M A, Uchida T, Takeda N, Miyamura T., J Virol. 1997 October; 71(10):7207-13. Essential elements of the capsid protein for self-assembly into empty virus-like particles of hepatitis E virus. Li T C, Takeda N, Miyamura T, Matsuura Y, Wang J C, Engvall H, Hammar L, Xing L, Cheng R H. J Virol. 2005 October; 79(20):12999-3006. Niikura M et al, Chimeric recombinant hepatitis E virus-like particles as an oral vaccine vehicle presenting foreign epitopes. Virology 2002; 293: 273-280). In one embodiment, the capsid proteins are modified capsid proteins and the VLPs derived therefrom are cysteine/lysine modified HEV VLPs. For example, the modified capsid proteins contain one or more cysteine/lysine residues in a surface variable loop of HEV ORF2, or a portion thereof.

Various expression systems can be used to express the capsid proteins of the present invention. Examples of expression systems useful for the production of virus-like particles of the present invention include, but are not limited to, bacterial expression system (e.g., E. coli), insect cells, yeast cells and mammalian cells. Preferred expression system of the present invention includes baculovirus expression systems using insect cells. General methods, for example, for handling and preparing baculovirus vectors and baculoviral DNA, as well as insect cell culture procedures, are outlined in A Manual of Methods for Baculovirus Vectors and Insect Cell Culture Procedures.

The capsid proteins of the present invention can be cloned into the baculovirus vector, and used to infect appropriate host cells (see, for example, O'Reilly et al., “Baculovirus Expression Vectors: A Lab Manual,” Freeman & Co. 1992.). An insect cell line (e.g., Sf9 or Tn5) can be transformed with a transfer vector containing polynucleic acids which encodes the capsid proteins of the invention. The transfer vector includes, for example, linearized baculovirus DNA and a plasmid containing the desired polynucleotides. The host cell line may be co-transfected with the linearized baculovirus DNA and a plasmid in order to make a recombinant baculovirus.

Purification of the virus-like particles of the present invention can be carried out according to the standard technique in the art (See, Li T C, et al., J Virol. 1997 October; 71(10):7207-13. Li T C, et al., J Virol. 2005 October; 79(20):12999-3006. Niikura M et al, Virology 2002; 293: 273-280). The purified VLPs are then resuspended in a suitable buffer.

In some embodiments, the modified capsid proteins or VLPs derived therefrom can be chemically conjugated to one or more bioactive agents. For example, one or more cysteine/lysine residues of the capsid proteins can be acylated, alkylated, arylated, succinylated, or oxidized using methods known in the art. In some cases, the one or more cysteine/lysine residues can be conjugated using a maleimide functional group to covalently conjugate a bioactive agent to the thiol moiety of the cysteine or lysine. In some cases, the bioactive agent can be modified to introduce a maleimide functional group using CLICK chemistry. For example, an alkyne derivative of the bioactive agent can be contacted with a maleimide-azide in the presence of CuSO₄ and ascorbic acid to produce a maleimide bioactive agent. The maleimide can then be contacted with the one or more cysteines/lysines of the modified capsid protein to covalently link the two molecules. In some cases, the conjugating is performed on capsid protein that is not assembled into a VLP (e.g., in the presence of EDTA, EGTA, and/or a reducing agent such as DTT or betamercaptoethanol). In some cases, the conjugating is performed on capsid protein that is assembled into a VLP.

### C. Encapsulation of Bioactive Agents

Another aspect of the invention relates to the encapsulation of one or more bioactive agents in HEV virus-like particles (e.g., cysteine/lysine modified HEV VLPs) (See, DNA vaccine-encapsulated virus-like particles derived from an orally transmissible virus stimulate mucosal and systemic immune responses by oral administration, Gene Therapy 2004. 11, 628-635. S Takamura, M Niikura, T-C Li, N Takeda, S Kusagawa, Y Takebe, T Miyamura and Y Yasutomi). Any standard technique in the art can be used to encapsulate a heterologous nucleic acid, protein, polypeptide, chemotherapeutic, imaging agent, nanoparticle, etc. into the VLPs of the present invention. An exemplary bioactive agent is insulin, either in the protein form or in the nucleic acid form. The general procedure involves (1) disassembling the VLPs formed by the capsid protein according to the present invention; and (2) reconstructing the VLPs in the presence of the bioactive agent. A skilled artisan would recognize that it is preferred to have purified VLPs before the encapsulation procedure. It is particularly preferred to have the VLPs depleted of, or substantially depleted of, any undesired materials (e.g., nucleic acids) before the encapsulation procedure.

Disassembly of VLPs can be carried out using any standard technique in the art. Reconstituted virus-like particle can be produced under physiological conditions (See, US Patent Publication No.: 20080131928). Often, disassembly of virus-like particles requires an agent to disrupt the assembly of VLPs, such as a reducing agent or a chelating agent (See, US Patent Publication No.: 20040152181). A skilled artisan would recognize that factors and conditions that affect assembly and disassembly include: pH, ionic strength, posttranslational modifications of viral capsid proteins, disulfide bonds, and divalent cation bonding, among others. For example, the importance of cation bonding, specifically calcium, in maintaining virion integrity has been shown for polyomavirus (Brady et al., J. Virol, 23:717-724, 1977), and rotovirus (Gajardo et al., J. Virol, 71:2211-2216, 1997). Also, disulfide bonds appear to be significant for stabilizing polyomavirus (Walter et al., Cold Spring Har Symp. Quant. Biol, 39:255-257, 1975; Brady et al., J. Virol, 23:717-724, 1977); and SV40 viruses (Christansen et al., J. Virol, 21:1079-1084, 1977). Also, it is known that factors such as pH and ionic strength influence polyomavirus capsid stability, presumably by affecting electrostatic interactions (Brady et al., J. Virol, 23:717-724, 1977; Salunke et al., Cell, 46:895-904, 1986; Salunke et al., Biophys. J, 56:887-900, 1980). Also, it is known that post-translational modifications of some viral capsid proteins may affect capsid stability and assembly, e.g., glycosylation, phosphorylation, and acetylation (Garcea et al., Proc. Natl. Acad. Sci. USA, 80:3613-3617, 1983; Xi et al., J. Gen. Virol, 72:2981-2988, 1991). Thus, there are numerous interrelated factors which may affect capsid stability, assembly and disassembly.

Preferably, the VLPs of the present invention is disassembled by the removal of calcium ions (See, Touze A, Coursaget P. In vitro gene transfer using human papillomavirus-like particles. Nucleic Acids Res 1998; 26:1317-1323; Takamura et al., DNA vaccine-encapsulated virus-like particles derived from an orally transmissible virus stimulate mucosal and systemic immune responses by oral administration. Gene Therapy 2004; 11:628-635). According to the present invention, a reducing agent or a chelating agent or both are used to disassemble the VLPs. Various reducing agents can be used. Preferred embodiments of the reducing agents include, but are not limited to, dithiothreitol (DTT). Various chelating agents can be used, e.g., ethylene glycol tetraacetic acid (EGTA) or ethylenediaminetetraacetic acid (EDTA). Examples of VLP disassembly conditions include, but are not limited to, the following: purified VLPs were disrupted by incubation of a buffer containing 50 mM Tris-HCl (pH 7.5), 150 mM NaCl, 1 mM EGTA and 20 mM dithiothreitol for 30 minutes.

A skilled artisan would also recognize that complete disassembly of the VLPs is not required, although preferred, to encapsulate a bioactive agent. An artisan would also recognize that, on other occasions, it is preferred to have partial disassembly of the VLPs. According to the present invention, the conditions for the partial disassembly of the VLPs can be controlled to still allow efficient encapsulation of a bioactive agent. Partial disassembly of the VLPs can be achieved by treatment of VLPs with reducing agents alone (e.g., 20 mM DTT) (Sapp et al, J. Gen. Virol., 76:2407-2412, 1995.). According to the present invention, once the VLPs are disassembled completely or partially, encapsulation of a bioactive agent can be carried out by reassembling the VLPs in the presence of the bioactive agent. In some cases, it can be advantageous to utilize a bioactive agent having a net negative charge to enhance encapsulation. For example, nucleic acids have a net negative charge and can be preferentially encapsulated as compared to compounds that have a positive or neutral charge.

In some embodiments of the present invention, reassembly of the VLPs is achieved by re-supplementation of calcium ions to the disrupted VLPs. Alternatively, reassembly of the VLPs is achieved by removal of the reducing agents or the chelating agents. Optionally, factors such as pH and ionic strength, other factors described in the present invention, can be adjusted to achieve efficient reassembly of the VLPs and efficient encapsulation of the bioactive agent.

In some embodiments, encapsulation is performed as follows: following 30 min of incubation at room temperature, a bioactive agent in 50 mM Tris-HCl buffer (pH 7.5) and 150 mM NaCl is added to the disrupted VLP preparation. The disrupted VLP preparation is then refolded by incubation for 1 h with increasing concentrations of CaCl₂ up to a final concentration of 5 mM. VLPs are pelleted by ultracentrifugation and resuspended in 10 mM potassium-MES buffer (pH 6.2). To estimate the amounts of encapsulated agent, refolded and purified VLPs are purified from any unencapsulated bioactive agent and disrupted with EGTA (1 mM). Absorbance of the supernatant, or other suitable methods can be used for detection of the bioactive agent.

In some embodiments, the bioactive agent (e.g., insulin protein or insulin-encoding nucleic acid) or imaging agent to be encapsulated is conjugated to an encapsidation signal. For example, an RNA element corresponding to codons 35-59 of HEV open reading frame 1 is a powerful encapsidation signal, allowing specific interaction in vitro with HEV capsid protein, including truncated and/or cysteine/lysine modified versions of HEV ORF2 VLP as described herein. To use VLP as a carrier for therapeutic or imaging agents, chemical linkers (e.g., LC-SPDP or aptamer, telodendrimers) that tag the agent (e.g., chemotherapeutic) with an HEV encapsidation signal like the foregoing RNA element can be used prior to the capsid self-assembly.

In some embodiments, a detectable label (imaging agent) is encapsulated. The detectable label can be a moiety renders a molecule to which it is attached to detectable by a variety of mechanisms including chemical, enzymatic, immunological, or radiological means. Some examples of detectable labels include fluorescent molecules (such as fluorescein, rhodamine, Texas Red, and phycoerythrin) and enzyme molecules (such as horseradish peroxidase, alkaline phosphatase, and β galactosidase) that allow detection based on fluorescence emission or a product of a chemical reaction catalyzed by the enzyme.

Radioactive labels involving various isotopes, such as ³H, ¹²⁵I, ³⁵S, ¹⁴C, or ³²P, can also be attached to appropriate molecules to enable detection by any suitable methods that registers radioactivity, such as autoradiography. See, e.g., Tij ssen, “Practice and Theory of Enzyme Immunoassays,” Laboratory Techniques in Biochemistry and Molecular Biology, Burdon and van Knippenberg Eds., Elsevier (1985), pp. 9 20. An introduction to labels, labeling procedures, and detection of labels can also be found in Polak and Van Noorden, Introduction to Immunocytochemistry, 2d Ed., Springer Verlag, NY (1997); and in Haugland, Handbook of Fluorescent Probes and Research Chemicals, a combined handbook and catalogue published by Molecular Probes, Inc. (1996). Further detectable labels include, but are not limited to, superparamagnetic labels (e.g., ferrite), contrast enhancing reagents (e.g., MRI contrast agents), atom-clusters (e.g., gold clusters), and the like. The conjugation of monodispersed gold cluster onto the modified capsid protein, e.g., onto cysteine/lysine residue(s) including the artificially introduced cysteine/lysine residue(s) in the modified capsid protein, can be performed according to the methods known in the art and described in various publications[18].

In some embodiments, a bioactive agent is encapsulated. In some cases, the bioactive agent is a chemotherapeutic. Suitable chemotherapeutics include, but are not limited to, cytotoxic drugs. Examples of cytotoxic drugs which may be used in the present invention include: alkylating drugs, such as cyclophosphamide, ifospfamide, ehlorambucil, melphalan, busulfan, lomustine, carmustine, chlormethhine (mustine), estramustine, treosulfan, thiotepa, mitobronitol; cytotoxic antibiotics, such as doxorubicin, epirubicin, aclarubicin, idarubicin, daunorubicin, mitoxantrone (mitozantrone), bleomycin, dactinomycin and mitomycin; antimetabolites, such as methotrexate, capecitabine; cytarabine, fludarabine, cladribine, gemcitabine, fluorouracil, raltitrexed (tomudex), mercaptopurine, tegafur and tioguaninc; vinca alkaloids, such as vinblastine, vincristine, vindesine, vinorelbine and etoposide; other neoplastic drugs, such as amsacrine, altetarmine, crisantaspase, dacarbazine and temozolomide, hydroxycarbamide (hydroxyurea), pentostatin, platinum compounds including: carboplatin, cisplatin and oxaliplatin, porfimer sodium, procarbazine, razoxane; taxanes including: docetaxel and paclitaxel; topoisomerase I inhibitors including inotecan and topotecan, trastuzumab, and tretinoin. In some cases, one or more of the foregoing imaging agents and/or bioactive agents, or a combination thereof, can additionally or alternatively be conjugated to a cysteine or lysine (e.g., recombinantly introduced cysteine or lysine) in a P-domain surface variable loop or C-terminus via a thiol linkage. In some cases, one or more of the foregoing imaging agents and/or bioactive agents, or a combination thereof, can additionally or alternatively be conjugated to a second cysteine or lysine (e.g., recombinantly introduced cysteine or lysine) in a P-domain surface variable loop or C-terminus via a thiol linkage.

In some embodiments, insulin is the bioactive agent encapsulated in the HEV VLP construct of this invention. Insulin in the form of a biologically active polypeptide (which may include optional post-translational modification, such as glycosylation, PEGylation, or substitution of one or more artificial amino acid analogues including D-amino acids, etc.) is used in some cases, whereas in other cases, insulin is in the form of a polynucleotide sequence (e.g., cDNA) encoding the insulin and/or proinsulin protein, for example, the insulin-encoding nucleic acid is a human insulin gene expression construct in a TAlm vector[12]. The insulin protein may be recombinant or it may be isolated from a natural source. It may be a human insulin or derived from other animals such as bovine, porcine, feline, or canine animals. It may be proinsulin. Different forms of insulin can be used: rapid-acting (Aspart: Novolog; Glulisine; Apidra; Lispro: Humalog); short-acting (Regular: Humulin, Humulin R, Novolin); intermediate-acting (NPH: Humulin N, Novolin N); intermediate to Long-acting (Detemir); long-acting (e.g., Glargine). Furthermore, the bioactive agent may be an analogue of insulin, such as a commercial insulin analog marketed as Levemir; or insulin glargine, which is a long-acting basal insulin analogue and marketed under the names Lantus. Additionally, the bioactive agent may be a combination of an insulin and glucagaon like peptide (GLP-1) receptor or other drugs. Examples of GLP-1 receptor agonists include liraglutide (Victoza, Saxenda), lixisenatide (Lyxumia), albiglutide (Tanzeum), dulaglutide (Trulicity), and semaglutide (Ozempic). Suitable forms or combinations of insulin include but are not limited to insulin glargine; insulin lispro; insulin aspart; insulin detemir; insulin (human); insulin aspart+insulin aspart protamine; insulin glulisine; insulin (human)+insulin isophane [INN]; insulin aspart+insulin degludec; insulin aspart+insulin isophane [INN]; insulin degludec+liraglutide; insulin glargine+lixisenatide; insulin human+insulin isophane [INN]; insulin isophane [INN]+insulin neutral; insulin isophane human [INN]+insulin human; insulin (bovine); insulin degludec; insulin human zinc; insulin isophane [INN]; insulin isophane human [INN]; insulin neutral; insulin human+insulin isophane human [INN]; insulin neutra+insulin isophane [INN]; insulin (porcine); insulin, neutral; protamine zinc insulin; insulin; insulin tregopil [INN]; insulin human+proinsulin human; insulin glargine+insulin lispro; insulin human+pramlintide acetate; dulaglutide; dulaglutide+insulin glargine; exenatide+insulin lispro; insulin glargine+liraglutide; insulin lispro+pramlintide; efpeglenatide [INN]; insulin human+pramlintide; exenatide+insulin human; insulin lispro+insulin lispro protamine; clioquinol [INN]+insulin human; insulin glargine+insulin glulisine; and insulin I 131. Further, various peptidyl and non-peptidyl insulin mimetics such as those described in by Nankar et al. (Drug Discovery Today, Volume 18, Issues 15-16, August 2013, Pages 748-755) may be used as bioactive agents for encapsulation in HEV VLPs.

The size of the VLPs can vary when different constructs of the capsid protein are used. For example, the N-terminal portion of the capsid protein can be adjusted to increase or decrease the size and encapsulation capacity of the VLPs. In some embodiments of the invention, in constructing the HEV VLP, a portion of HEV ORF 3 protein fused to the N-terminal of a portion of HEV ORF 2 proteins is utilized to adjust the size of the VLPs. Typically, the HEV VLP is formed from a portion of HEV ORF2 having at least residues 112-608 of HEV ORF 2.

### D. Pharmaceutical Compositions, Formulations, and Administration

The present invention also provides pharmaceutical compositions or physiological compositions comprising an HEV VLP formed by a modified capsid protein encapsulating an bioactive agent such as insulin in the form of a protein or nucleic acid. Such pharmaceutical or physiological compositions also include one or more pharmaceutically or physiologically acceptable excipients or carriers. Pharmaceutical compositions of the invention are suitable for use in a variety of drug delivery systems. Suitable formulations for use in the present invention are found in Remington's Pharmaceutical Sciences, Mack Publishing Company, Philadelphia, PA, 17th ed. (1985). For a brief review of methods for drug delivery. See Langer, Science 249: 1527-1533 (1990).

The compositions of the present invention can be administered to a host with an excipient. Excipients useful for the present invention include, but are not limited to, vehicles, binders, disintegrants, fillers (diluents), lubricants, glidants (flow enhancers), compression aids, colors, sweeteners, preservatives, suspending/dispersing agents, film formers/coatings, flavors and printing inks.

One advantage of the present invention is that the compositions of the present invention are suitable for oral delivery. Because the HEV VLP of this invention is capable of targeting the liver cells, cite-specific delivery of insulin can be effective achieved. Also, as a result of the modification of the capsid protein the HEV VLP of this invention is stable in an acidic environment and resistant to digestion in the gastrointestinal tract, it is suitable for oral delivery of insulin. The gold nanocluster conjugated to the cysteine or lysine residue(s), especially those engineered into the surface of a modified capsid protein in some embodiments of the present invention, further enhances the stability, bioavailability, and delivery efficiency of the HEV VLP. Thus, oral delivery of the compositions of the present invention can effective provide therapeutic benefits for patients suffering from a condition of insulin insufficiency or dysregulation, such as type I or II diabetes as well as the associated symptoms. The HEV VLP of this invention may be formulated in the form of a solid (e.g., powder) or a liquid such that it may be used as a supplement to ordinary food or beverage items for consumption in daily life.

Additionally, the compositions of the present invention may also be formulated for mucosal delivery, such as delivery to the buccal or labial mucosa or the respiratory tract mucosa, including the nasal mucosa.

The pharmaceutical compositions of the present invention can be administered by various routes, e.g., oral, subcutaneous, transdermal, intradermal, intramuscular, intravenous, or intraperitoneal. The preferred routes of administering the pharmaceutical compositions are oral delivery at daily doses of about 0.01-5000 mg, preferably 5-500 mg, of the HEV VLP. Oral administration is a preferred mode of administration, and the appropriate dose may be administered in the form of tablets, capsules, or as a supplement to food or beverage items in a single daily dose or as divided doses presented at appropriate intervals, for example as two, three, four, or more subdoses per day.

For preparing pharmaceutical compositions of the present invention, inert and pharmaceutically acceptable carriers are used. The pharmaceutical carrier can be either solid or liquid. Solid form preparations include, for example, powders, tablets, dispersible granules, capsules, cachets, and suppositories. A solid carrier can be one or more substances that can also act as diluents, flavoring agents, solubilizers, lubricants, suspending agents, binders, or tablet disintegrating agents; it can also be an encapsulating material.

In powders, the carrier is generally a finely divided solid that is in a mixture with the finely divided active component, e.g., a chimeric virus-like particles with an encapsulated nucleic acid. In tablets, the active ingredient (a chimeric virus-like particles with an encapsulated nucleic acid) is mixed with the carrier having the necessary binding properties in suitable proportions and compacted in the shape and size desired.

For preparing pharmaceutical compositions in the form of suppositories, a low-melting wax such as a mixture of fatty acid glycerides and cocoa butter is first melted and the active ingredient is dispersed therein by, for example, stirring. The molten homogeneous mixture is then poured into convenient-sized molds and allowed to cool and solidify.

Powders and tablets preferably contain between about 5% to about 70% by weight of the active ingredient. Suitable carriers include, for example, magnesium carbonate, magnesium stearate, talc, lactose, sugar, pectin, dextrin, starch, tragacanth, methyl cellulose, sodium carboxymethyl cellulose, a low-melting wax, cocoa butter, and the like.

The pharmaceutical compositions can include the formulation of the active compound with encapsulating material as a carrier providing a capsule in which the active component (with or without other carriers) is surrounded by the carrier, such that the carrier is thus in association with the compound. In a similar manner, cachets can also be included. Tablets, powders, cachets, and capsules can be used as solid dosage forms suitable for oral administration.

Liquid pharmaceutical compositions include, for example, solutions suitable for oral or parenteral administration, suspensions, and emulsions suitable for oral administration. Sterile water solutions of the active component (e.g., a chimeric virus-like particles with an encapsulated nucleic acid) or sterile solutions of the active component in solvents comprising water, buffered water, saline, PBS, ethanol, or propylene glycol are examples of liquid compositions suitable for parenteral administration. The compositions may contain pharmaceutically acceptable auxiliary substances as required to approximate physiological conditions, such as pH adjusting and buffering agents, tonicity adjusting agents, wetting agents, detergents, and the like. It is also expected that the HEV VLP may be in the form of tablets/capsules in prepackaged powder or concentrated liquid form as sold. This would be further added into food or beverage including water by the patient and then consumed by the patient. The HEV VLP can also be in liquid form and directly consumed without further dilution.

Sterile solutions can be prepared by suspending the active component (e.g., a chimeric virus-like particles with an encapsulated nucleic acid) in the desired solvent system, and then passing the resulting solution through a membrane filter to sterilize it or, alternatively, by dissolving the sterile compound in a previously sterilized solvent under sterile conditions. The resulting aqueous solutions may be packaged for use as is, or lyophilized, the lyophilized preparation being combined with a sterile aqueous carrier prior to administration. The pH of the preparations typically will be between 3 and 9, more preferably from 5 to 8, and most preferably from 6 to 7.

The pharmaceutical compositions of the present invention can be administered for prophylactic and/or therapeutic treatments. In therapeutic applications, compositions are administered to a patient already suffering from a condition in an amount sufficient to prevent, cure, reverse, or at least partially slow or arrest the symptoms of the condition and its complications. An amount adequate to accomplish this is defined as a “therapeutically effective dose.” Amounts effective for this use will depend on the severity of the disease or condition and the weight and general state of the patient, but generally range from about 0.1 mg to about 2,000 mg of the composition per day for a 70 kg patient, with dosages of from about 5 mg to about 500 mg of the composition per day for a 70 kg patient being more commonly used.

In prophylactic applications, pharmaceutical compositions of the present invention are administered to a patient susceptible to or otherwise at risk of developing a disease or condition, such as diabetes, in an amount sufficient to delay or prevent the onset of the symptoms. Such an amount is defined to be a “prophylactically effective dose.” In this use, the precise amounts of the composition again depend on the patient's state of health and weight, but generally range from about 0.1 mg to about 2,000 mg of the inhibitor for a 70 kg patient per day, more commonly from about 5 mg to about 500 mg for a 70 kg patient per day.

Single or multiple administrations of the compositions can be carried out with dose levels and pattern being selected by the treating physician. In any event, the pharmaceutical formulations should provide a quantity of composition of the present invention sufficient to achieve an intended effect in the patient, either therapeutically or prophylactically.

### EXAMPLES

The following examples are provided by way of illustration only and not by way of limitation. Those of skill in the art will readily recognize a variety of non-critical parameters that could be changed or modified to yield essentially the same or similar results.

### Example 1

### Oral Insulin Delivery by HEVNP

### I. Background

For the past eight decades, subcutaneous injection (SC) has been the main route used for supplementing the suboptimal insulin secretion for administering insulin as a treatment for diabetes mellitus. Although this method is effective, SC injections are painful, inconvenient, and carries high risk of infections leading to poor patient compliance. The insulin encapsulated Hepatitis E virus nanoparticle (HEVNP), composed of the noninfectious Hepatitis E viral capsid, is expected to deliver insulin from the gastrointestinal (GI) tract to the liver after ingestion. HEVNP can be the answer to the long search of effective and efficient means to administer insulin orally, the most preferred route of drug delivery with highest patient compliance.

### II. Structurally Stabilized HEVNPs for Oral Delivery of Insulin

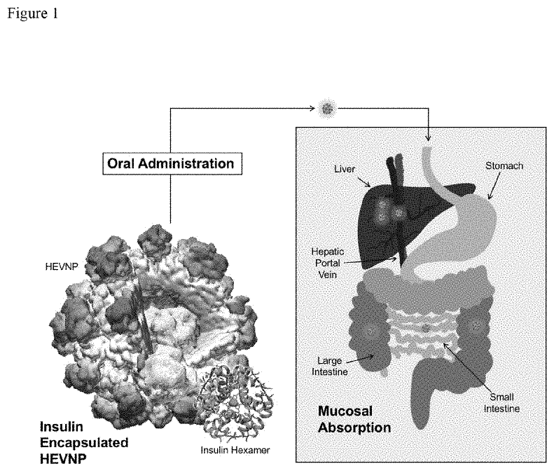

From the physiological point of view, orally administered insulin has therapeutic advantages in the management of hepatic glucose production because of its potential to mimic the endogenous insulin secretion pathway[4]. Following HEV's natural route of infection, insulin encapsulated HEVNP can travel through gastrointestinal tract through the portal vein, and to the liver (FIG. 1). In contrast, parenteral or inhaled insulin is absorbed directly into the peripheral circulation, bypassing hepatic extraction, thus failing to restore the portal-peripheral insulin gradient and physiologic hepatic insulinization. In addition, these routes expose peripheral targets to greater insulin concentrations relative to the liver, predisposing patients to a high risk of hypoglycemia, and the deleterious effects of hyperinsulinemia[4].

Hepatitis E Virus Nanoparticles (HEVNPs), derived from a modified form of the Hepatitis E Virus (HEV) capsid protein, are non-infectious, self-assembling capsids that lacks the viral genome and are capable of cell-binding and entry. Because HEV has evolved for orally mucosal transmission, the assembled capsid protein is similarly stable in proteolytic and acidic mucosal conditions[13]. High-yield HEVNP production has been achieved through the insect cell expression system via the baculovirus vector. Because of their proteolytic stability, self-assembled HEVNPs can be extracted and purified directly from cell supernatant, substantially reducing necessary purification steps. Moreover, HEVNPs possess a surface exposed protrusion domain (P domain) connected through a flexible hinge to a stable icosahedral base. The P domain can be modified without compromising the base icosahedral structure, by inserting a foreign peptide via genetic engineering[13] or chemical conjugation [14]. Three well-exposed surface variable loops on the P domain and the C terminal of the HEV capsid protein (CP), coded by open reading frame 2 (ORF2), are designed as genetic-engineered and/or chemical conjugation sites for at least one or more bioactive agents[14, 15].

Targeted drug delivery to specific organs and cell compartments has been proposed to reduce side effects to non-specific organs/cells. HEVNP has been proposed as a cell-targeted delivery system because its surface-exposed cysteine or lysine residue can adopt synthetic ligands for tissue targeting[14, 15]. Its capability of orally delivering gene has been proven in prior research when HEVNP orally delivered plasmid cDNA to the small intestine epithelial cells for transient expression of insulin and/or proinsulin [16, 17]. An in vivo biodistribution assay of HEVNP in a mouse model with Far Infrared (FIR) imaging (data not shown) indicated that orally delivered HEVNPs accumulate in the liver-even without specific liver-targeting ligand.

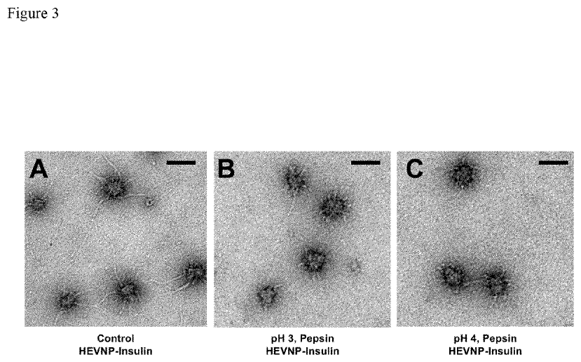

Encapsulation by HEVNP is based on charge interactions such that negatively-charged nucleic acids and nano-sized protein/small molecules can be packaged for therapeutic applications. HEVNP can encapsulate the commercial Insulin analog, Insulin detemir by Levemir (website: levemir.com), at the size ˜52nm (FIG. 3). Considering the pharmaceutical toxicology of HEVNP, it is composed of copies of single capsid protein ORF2 and is biodegradable. In additional, HEVNP can encapsulated insulin or proinsulin cDNA for oral gene delivery. The pancreatic β cell and/or liver targeted capability can be added by inserting specific cell-targeting ligand onto HEVNP's protrusion domains through overnight chemically conjugation or a time consuming but cost effective genetic engineering. HEVNP's tissue targeting capability makes it an advantageous oral delivery carrier for transporting insulin genes to the pancreas and/or liver, allowing temperous on-site insulin expression.

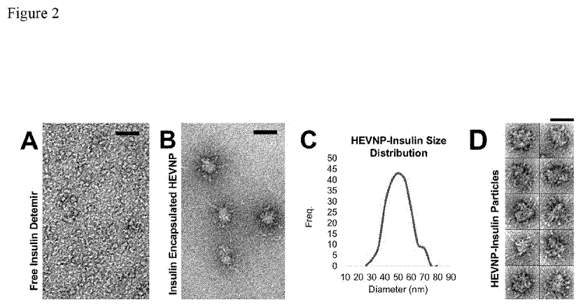

The concept of using HEVNP as an orally delivery carrier has not only been proven by prior research as mentioned but also supported by in vitro stability studies. An in vitro stability assay at different pH and Pepsin digestion test (unpublished data) shows that insulin encapsulated HEVNP can survive in a pH 3 environment with Pepsin digestion for 5 min (FIG. 2). The HEVNP contains a modified ORF2 capsid protein having one or more modifications described in WO2015/179321, U.S. Pat. No. 8,906,862, and U.S. Pat. No. 8,906,863. The bioavailability of encapsulated insulin can be further assured via drinking before a meal to avoid the harsh digestive environment in stomach. Additionally, bioavailability can be stabilized by chemically conjugating monodispersed gold nano-clusters (AuNCs) onto HEVNP's five-fold symmetry region [18]. Furthermore, the AuNC has been proposed as in vivo imaging reagent due to its FIR detectable signal, which could penetrate deep tissue [19]. The combination of HEVNP's functions, including insulin encapsulation, insulin/proinsulin cDNA encapsulation and tissue/cell targeting via surface conjugation capability, makes it an ideal oral delivery system of insulin itself or gene of insulin for treating diabetes. The delivery system improves patient compliance by eliminating the use of needles.

The present invention resides in an HEVNP platform, which has (1) a tissue/cell targeting ligand (especially a ligand capable of specifically directing the HEVNP to liver cells) conjugating onto its surface to enhance its absorption, and (2) insulin (in the form of either an insulin polypeptide or a polynucleotide sequence encoding insulin) encapsulated in its interior for drug/gene delivery. The HEVNP is constructed in accordance with prior disclosures by the present inventors including U.S. Pat. No. 8,906,862, U.S. Pat. No. 8,906,863, and WO2015/179321.

### III. Summary

HEVNP, a REV-derived nano-capsule deprived of viral infectivity, retains essential features of HEV that include gastrointestinal stability, target cell binding, and cell entry. Combined with its ability to disassemble/reassemble in vitro, HEVNP has been proposed as an attractive oral delivery nano-capsule via drinking. Encapsulation by HEVNP is an electrostatic interaction between payloads, and capsid proteins such that negatively-charged nucleic acids and nano-sized protein/small molecules can be packaged for therapeutic applications. In addition to the encapsulation of insulin for oral delivery to the liver via the GI tract, the insulin gene can also be encapsulated. If necessary, pancreatic β cell and/or liver targeted capability can be added by inserting specific cell targeting ligand onto HEVNP's protrusion domains through overnight chemical conjugation or time consuming but cost effective genetic engineering. Thus, HEVNP is equipped to be a cell-targeted, gene delivery carrier that can deliver the insulin gene to the pancreas and transiently express insulin on site. The insulin encapsulated HEVNP is expected to deliver insulin from the gastrointestinal tract to the liver by oral administration, the preferred route of drug administration.

In a combined-modality therapy scheme a diabetic patient is treated with two or more diabetes treatments that improve the control of blood glucose levels. Multiple modalities of diabetes treatment can be offered by HEVNPs by switching payloads between insulin in insulin/proinsulin polypeptide form and insulin/proinsulin eDNA. form to achieve different in vivo kinetics of delivered insulin. Another level of modality comes from conjugating different tissue/cell targeting ligands on the protrusion domain of HEVNPs. The combination of these multi-modality treatments, by orally delivering insulin encapsulated HEVNPs and/or the HEVNP containing insulin/proinsulin cDNA, can be an alternative diabetes treatment to needle injection.

### IV. Materials and Methods

### 1. HEVNP Encapsulation of Insulin

1.1. Disassembly of the HEVNP
1.1.1. Disassemble the HEVNPs in 20mM DTT, 10mM EDTA, 0/N at 4C
1.1.2. Dialysis the disassembled HEVNPs against 50mM Tris, pH7.5, 150mM NaCl at RT>1H
1.1.3. Check by TEM, Protein conc. Measurement by spectrophotometry
1.2. Encapsulation of insulin into HEVNP
1.2.1. Mix the disassembled HEVNP with insulin in 50 mM Tris, pH7.5, 150 mM NaCl, add CaCl₂ to make final con. 2-5 mM CaCl₂. O/N at 4C
1.2.2. Go through size exclusion column to remove free insulin.
1.2.3. Collect the fractions and measure protein conc. By spectrophotometer
1.2.4. Check the Insulin encapsulated HEVNP by TEM

### 2. HEVNP Characterization

2.1. Record A280 nm reading and A260/A280 nm ratio using spectrophotometer. The molar extinction coefficient of HEVNP ORF2 is 60,280, which is equivalent to 1.019 x protein absorbance value at 280 nm. This is so close to 1:1 that the concentration of HEVNPs can be represented by the protein concentration measurement at A280 nm by spectrophotometer. Considering the building block of HEVNP, ORF2, with its molecular weight at 53.318 kDa:

Molar concentration of ORF2 (M)=A280 nm (^{mg/ml)/}53,318g/mol

For example: The HEVNP has concentration of 1 mg/mL from spectrophotometer measurement at 280 nm, which is equivalent to 18.8 μM of ORF2. (Each ORF2 contains 1 Cys site and 1 Lys site for chemical conjugation.)
2.2. Prepare SDS PAGE 4-12% Bis-Tris Protein Gels, 1.0 mm, 17-well according to user manual¹⁴:
2.2.1. Add 2μl of 4× loading buffer to 6 μl of protein sample. Incubate the sample mixture in heat block for 10 min at 100 ° C. to denature the protein. Load protein samples onto a NuPAGE gel set up.
2.2.2. Run SDS-PAGE by setting the DC power supply at 100 V for 10 min, then 150 V for 45 min until the samples run to about 1 cm above the bottom of the gel.
2.2.3. Stain the SDS PAGE gel with Coomassie blue, (0.25% (w/v) Coomassie Brilliant Blue R250, 30% (v/v) methanol, 10% (v/v) acetic acid), for 1 h.
2.2.4. After the stain procedure, remove Coomassie blue stain and apply distaining buffer (30% (v/v) methanol, 10%(v/v) acetic acid) onto protein gel for >12 h at room temperature.
2.2.5. Document the gel under white light to confirm the presence of HEVNP ORF2 at 52 kDa band.
2.3. Observe HEVNPs using TEM
2.3.1. Prepare or dilute HEVNP samples to 0.5-2 mg/mL with 10 mM MES pH6.2 for TEM imaging.
2.3.2. Ionize carbon-coated grids with 40 mA glow discharge for 30 seconds to produce hydrophilic carbon surface. The glow discharge equipment can be EMS glow discharger. The hydrophilic carbon surface of grids can only last for 30 min after glow discharge treatment.
2.3.3. Hold in tweezers and add 2 μL of HEVNP sample to grid, wait for 15-30 seconds, and blot with filter paper.
2.3.4. Immediately wash grid with ddH₂0 and blot with filter paper.
2.3.5. Immediately add 2 μL of 2% uranyl acetate to grid, wait 15 seconds, then blot with filter paper. Dry the sample grids by putting them in electronic dehumidify dry cabinet for overnight.
2.3.6. Transfer the grid into transmission electron microscope (TEM) and image at 10K to 80K magnification. HEVNPs appear in TEM as empty icosahedral proteins ˜27nm in diameter, due to the absence of viral RNA.
   3. Chemical Conjugation of HEVNPs with Biotin, tissue/cell Targeting Ligand and Fluorophores
3.1. One Step Conjugation of HEVNPs and Maleimide linked Biotin
3.1.1. Buffer change: Apply HEVNPs in mini dialysis units and dialysis against 0.01M PBS pH=7.4 at room temperature for 1 hour according to manufacturer's protocol (Zeba Spin Desalting Columns, 40K MWCO, 0.5mL). Transfer HEVNPs to 1.5 mL tubes and measure protein concentration at 280 nm using spectrophotometer.
3.1.2. Mix HEVNP at lmg/mL, which is equivalent to 18.8 μM of Cys reaction site (see details in step. 2.2.4), with equal amount of maleimide-Biotin (100 μM) in 0.01M PBS pH7.4 to make a 1:5 mole ratio and react O/N at 4° C. Remove unbound maleimide-biotin with 40K MWCO Spin Desalting column procedure according to manufacturer's protocol (Zeba Spin Desalting Columns, 40K MWCO, 0.5 mL).
3.1.3. Analyze samples through standard reducing SDS-PAGE (step 3.1).
3.1.4. Prepare Chemiluminescent Western Blot, HRP-linked Streptavidin. Capture chemiluminescent signal by X ray film (FIG. 2).
3.2. Two Step Tissue targeted ligand (RGD peptide) Conjugation to Surface Exposed Cysteine on HEV NPs.
3.2.1. Buffer change: Apply HEVNPs in mini dialysis units and dialysis against 0.01M PBS pH=7.4 at room temperature for 1 h. Transfer HEVNPs to 1.5mL tubes and measure protein concentration at 280 nm using spectrophotometer.
3.2.2. Add 650 μM maleimide-azide and 650 μM alkyne-LigandX in 0.01M PBS pH7.4 with 200 μM CuSO₄ and 1 mM ascorbic acid to form maleimide-linked LigandX (Mal-LigandX) at 650 μM. Incubate the mixture at 4° C. overnight.
3.2.3. Mix HEVNP at lmg/mL, which is equivalent to 18.8 μM of Cys reaction site (see details in step. 2.2.4), with about 10% volume of Mal-LigandX (650 μM) in 0.01M PBS pH7.4 to make a 1:3 molar ratio and react O/N at 4° C. Due to the relatively high concentration of maleimide-linked LXY30, the final concentration of reactants, such as CuSO₄, are reduced about 10 times after mixing to avoid their damage to HEVNPs. Another option is the Cu free conjugation method¹⁵.
3.2.4. Remove unbound maleimide-click-LigandX with 40K MWCO Spin Desalting column according to manufacturer's protocol (table of materials). Keep the LXY30-linked HEVNPs (LXY30-HEVNPs) at 4° C.
3.3. One Step Conjugation of LXY30-linked HEVNPs (LigandX-HEVNPs) and Cy5.5 NHS ester (NHS-Cy5.5)
3.3.1. Mix LigandX-linked HEVNPs (LigandX-VLPs) at 1 mg/ml, which is equivalent to 18.8 μM of Cys reaction site (see details in step. 2.2.4), with equal volume of Cy5.5 NHS ester (NHS-Cy5.5, 100 μM) in 0.01M PBS pH7.4 to make a 1:5 molar ratio and react O/N at 4° C.
3.3.2. Remove unbound Cy5.5-NHS by going through 40K MWCO Spin Desalting column procedure according to manufacturer's protocol (Zeba Spin Desalting Columns, 40K MWCO, 0.5 mL). Keep the RGD, Cy5.5-linked HEVNPs (RGD-HEVNP-Cy5.5) at 4° C.

### Example 2

### In Vivo Studies

### I. HEVNP Encapsulation Design

In the formulation, HEVNP can be formulated as a tablet, capsule, sprinkle powder, or liquid to be included in drinks. HEVNP subcomponents have been proven safe vaccines for human and animals. In contrast to other proposed enhancers of oral insulin administration, HEVNP capsules are enabled as a mucosa-focused delivery system with enhanced bioavailability for protein payloads like insulin through oral routes. Quaternary structure-based payloads are designed to utilize macromolecular attributes to extend the duration of actionable retention time.

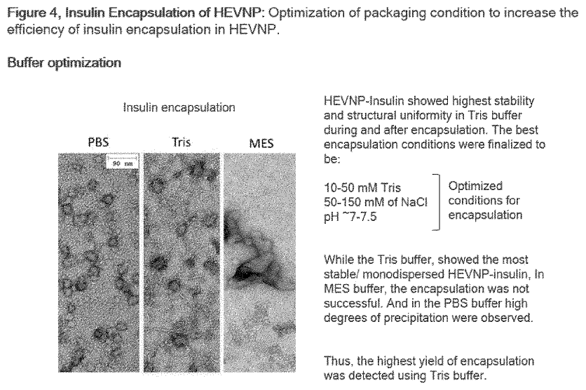

To optimize the encapsulation efficiency of insulin, multiple assays were carried out to examine the optimal conditions. As shown in FIG. 4, the encapsulation of insulin in HEVNP showed the highest stability and structural uniformity in Tris buffer during and after encapsulation. The optimal encapsulation conditions were narrowed down to 10-50 mM Tris, 0-150 mM NaCl, in a range of neutral pH. The MES buffer, in contrast, provided least the favorable condition for payload encapsulation while the PBS buffer produced high degrees of precipitation. As the Tris buffer offered stable and monodisperse HEVNP with protein payloads in solution, the highest yield of encapsulation was further identified in conditions using Tris buffers.

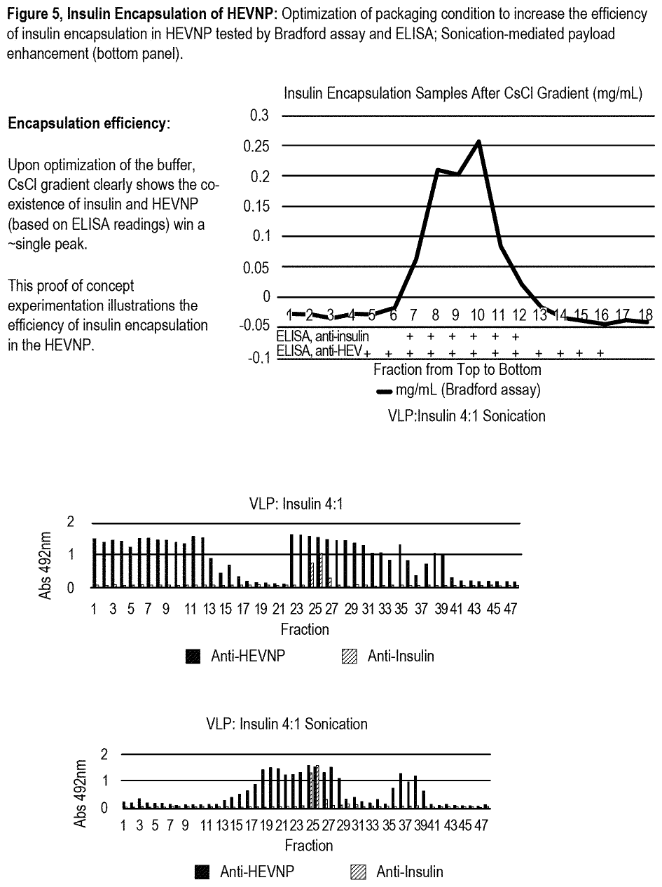

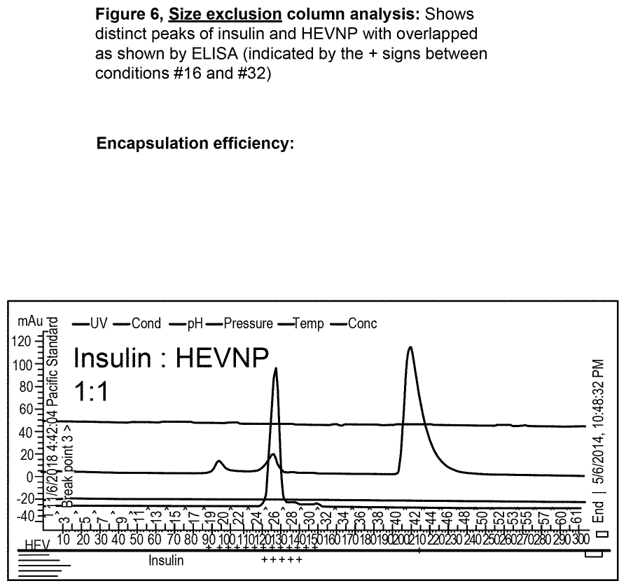

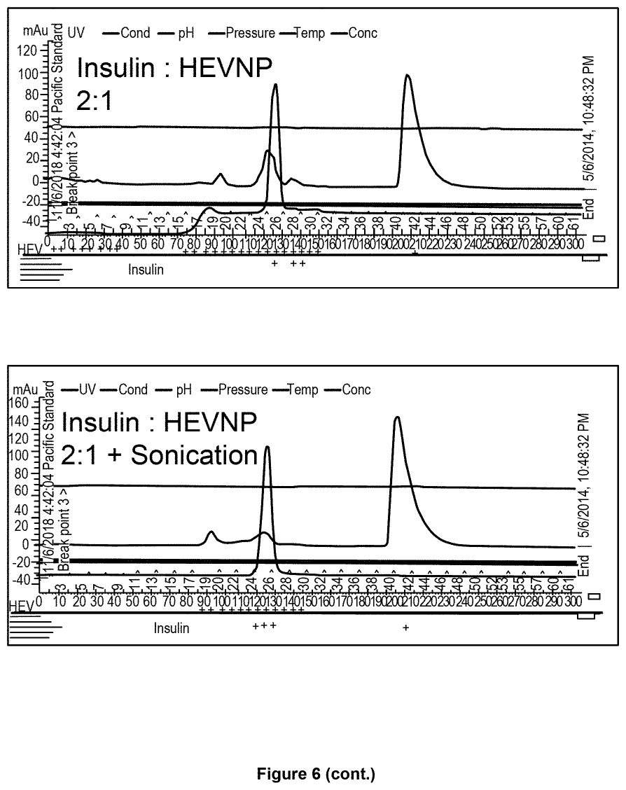

For the encapsulation, HEVNP subunits are incubated with corresponding molar ratios of protein payloads like insulin to gradually assemble the capsules with added calcium chloride in the system. The efficiency of insulin encapsulation was monitored and assessed in the following:
1. Cesium chloride density gradient separation; the coexistence of HEVNP and Insulin are shown by ELISA (against HEV and insulin) (FIG. 5)
2. Size exclusion column separation; the coexistence of HEVNP and Insulin are shown by (against HEV and insulin) (FIG. 6)

### II. HEVNP Encapsulation With Density Assessments

Upon optimization of the buffer, the CsCl gradient clearly shows the co-existence of insulin and HEVNP within a single peak of ELISA readings to illustrate the efficiency of insulin encapsulation in the HEVNP. The “+” indicates positive readout from ELISA and the coexistence of both HEV and insulin in fractions 6-13.

Size assessments identify novel conformations of HEVNP carrying insulin payloads

The SEC shows distinct peaks of insulin and HEVNP with overlapped as shown by ELISA (indicated by the + signs between fraction #16 and #32)

As indicated by the first peak (red peak), additional evidence to identify coexistence of insulin and HEVNP capsules, validated by ELISA assays following the specificity of anti-insulin and anti-HEVNP antibodies, respectively. Further encapsulation was systematically monitored further to identify sonication-mediated payload optimizations into the new forms of HEVNPs (FIG. 5 bottom panel): single peak (excluding the outlier fractions beyond 35), shown a unified peak with both insulin and HEV (validated by ELISA, absorbance reading at 492 nm).

### III. Prolonged HEVNP Shelf Life

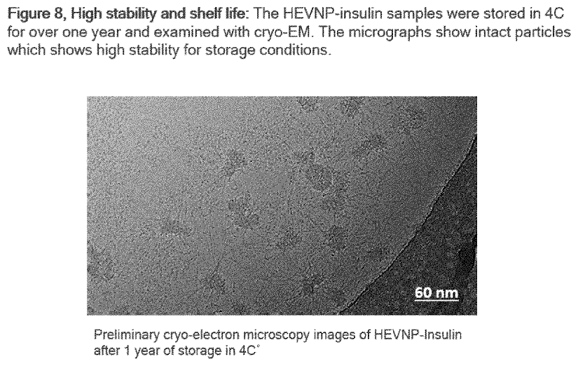

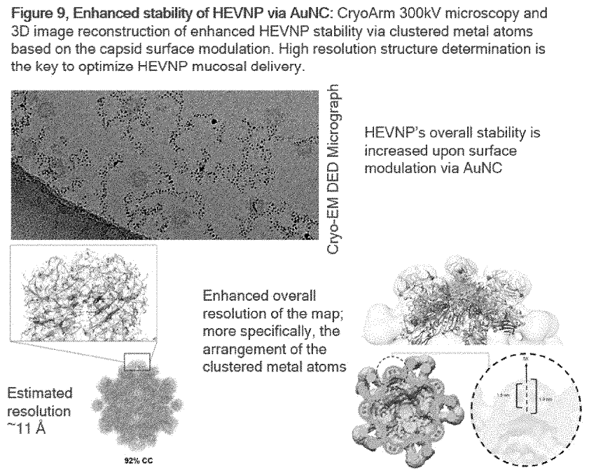

For an effective medicinal delivery system, high stability and shelf life of the product is critical. The HEVNP-insulin samples were stored in 4C for over one year and examined with cryo-EM. The micrographs show intact particles which show high stability for storage conditions. Cryo-electron microscopy was utilized to examine the HEVNP particle with encapsulated insulin detemir, as shown in FIG. 8.

### IV. Structural Characterization of HEVNP-Insulin:

Results from electron microscopy have been provided that indicate insulin encapsulation; however, the 2-dimensional distribution and 3-dimensional structural features of these nanoparticles are yet to be fully characterized. Using a combination of in-house protocols and commercially available image processing packages, a large dataset have been collected and analyzed to 1) statistically analyze particle distribution, and 2) determine the high-resolution 3D structure of insulin-encapsulated-HEVNP.

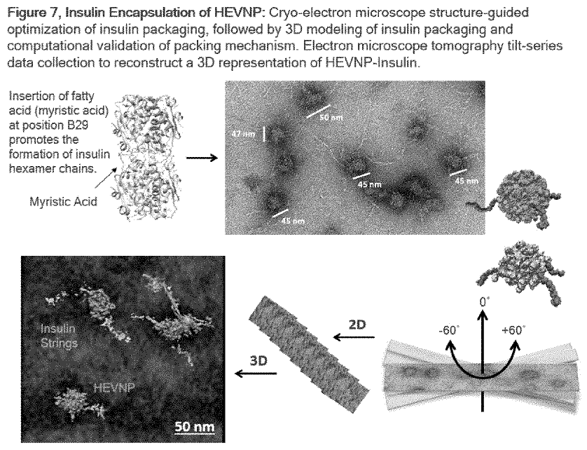

Assessments TEM images indicate a novel conformation of HEVNP-Insulin created with almost twice larger diameter ˜45 nm than that of our first generation of HEVNP (27 nm) previously filed. Within these HEVNP, the novel shapes and sizes appear optimal to carry insulin payloads with extruded strands of hexameric nodes visually accessible. Further 3D volume characterization was carried out by cryo-electron microscope to achieve structure-guided optimization of insulin packaging efficiency. This new generation of HEVNP confrontation was fulfilled by computational modeling to perfect the preload packaging. Electron 3D tomography with tilt-series data collected to reconstruct a 3D representation of HEVNP-Insulin was carried out with the digital segmentation to analyze the packing mechanism using a 200kV electron microscope (JEOL 2100F) from −60 to +60 degrees at 1-degree increments. The 3D reconstruction was carried out using the Simultaneous Iterative Reconstruction Technique method, which clearly shows the segmented strands of insulin extruding from the HEVNP, in FIG. 7.

### V. HEVNP Encapsulation Validated by Large and Small Animal Models:

The mice are assigned to be randomly assigned to one of 2 treatment groups and subjected to an insulin tolerance test as follows:
A. Orally administered insulin (HEVNP-encapsulated) at 0.1 U/mouse
B. Orally administered insulin (HEVNP-encapsulated) at 1 U/mouse

With an assumed 50% reduction in blood glucose concentrations after IP insulin administration vs. a mean of 25% reduction after oral insulin administration, with a standard deviation of 15% and the desired alpha error of 5% and 80% power, a subgroup of 10% mice are placed to detect a significant difference between groups.

Oral delivery is placed via gavage, using light isoflurane anesthesia and flexible gavage needles. 26G needles are used for the IP injection. Insulin and/or HEVNP are dissolved in 0.9% saline. If the oral insulin formulations are taken up across the mucosae to achieve the expected decrease in blood glucose levels.

In addition, 8-10 dogs modeled with diabetic disorders are subjected of trial as “patients” for the glucose monitoring measurements.

### VI. Whole-Animal Imaging to Trace the Encapsulated Payloads

In vivo optical imaging of mice using Cyanine-5.5 (Cy5.5)-labeled HEVNP has been previously demonstrated in Chen et al. “Chemically activatable viral capsid functionalized for cancer targeting.” Nanomedicine 11, no. 4 (2016): 377-390, where a breast tumor-targeting molecule (LXY30) was conjugated to an engineered cysteine arm and Cy5.5 linked to exposed lysine residues. The whole animal imaging demonstrated that HEVNPs with LXY30 will accumulate at the tumor site. Here, the surface of insulin-encapsulated HEVNP will be decorated with Cy5.5 NHS ester (Limiprobe) at a molar ratio of 300:1 (Cy5.5 to HEVNP) in buffer containing 0.01 M PBS, pH=7.2 at room temperature for 2 h, followed by incubation at 4° C. overnight. The free Cy5.5 NHS ester will then be removed by a 7000 MWCO desalting column (Zeba Spin Desalting Columns, Thermo Scientific). Cy5.5 has an excitation maximum at 682 nm, an emission maximum at 702 nm, and a molar extinction coefficient of 250,000 cm̂−1M̂−1.

Whole-animal imaging is followed to trace the HEVNP-insulin distribution with IVIS Spectrum for optical imaging (with resolving power of ˜20 μm-5 mm) and MicroXCT-200 for high-resolution CT (with resolving power of ˜1-20 μm). Oral insulin delivery route is through the mucosal lining of the GI after passing the stomach and towards the liver via the hepatic portal vein; thus, the accumulation in the liver where the nanoparticles release the insulin.

### VII. Molecular Features Described by Electron Microscopy

To study the HEVNP distribution on a cellular level, biopsy of the liver is conducted for embedding the tissue using high-pressure freezing methods and cryo-fixation. The extracted tissues are subject of light fixation with formaldehyde to be subsequently placed in specimen holders. The frozen tissues are then be fixed into resin blocks, which are then sectioned with an ultramicrotome and screened with transmission electron microscopy (TEM). HEVNP is tracked by the added contrast either by clusters of gold atoms by a 10 nm ferrite oxide particle. The electron-dense HEVNP particles provide sufficient contrast to be ID'ed by TEM.

High-pressure freezing and TEM preparation is to obtain high-resolution 3D images of the cellular level of ultrastructures with JEM 2100F electron microscope as described, see, e.g., Paavolainen et al., “Compensation of missing wedge effects with sequential statistical reconstruction in electron tomography.” PloS one 9, no. 10 (2014): e108978; Soonsawad et al., “Permeability changes of integrin-containing multivesicular structures triggered by picornavirus entry.” PloS one 9, no. 10 (2014): e108948; and Soonsawad et al.,“Structural evidence of glycoprotein assembly in cellular membrane compartments prior to Alphavirus budding.” Journal of virology 84, no. 21 (2010): 11145-11151.

All patents, patent applications, and other publications, including GenBank Accession Numbers, cited in this application are incorporated by reference in the entirety for all purposes.

### REFERENCES

1. Saaddine J B, Cadwell B, Gregg E W, Et Al. Improvements in diabetes processes of care and intermediate outcomes: United states, 1988-2002. Annals of Internal Medicine 144(7), 465-474 (2006).
2. Hoffman A, Ziv E. Pharmacokinetic considerations of new insulin formulations and routes of administration. Clinical pharmacokinetics 33(4), 285-301 (1997).
3. Owens D R. New horizons—alternative routes for insulin therapy. Nature reviews. Drug discovery 1(7), 529-540 (2002).
4. Arbit E, Kidron M. Oral Insulin Delivery in a Physiologic Context: Review. J Diabetes Sci Technol 11(4), 825-832 (2017).
5. Carino G P, Mathiowitz E. Oral insulin deliverylAbbreviations: GI, gastrointestinal; IDDM, insulin-dependent diabetes mellitus; IU, international units; NIDDM, non-insulin-dependent diabetes mellitus; PIN, phase inversion nanoencapsulation; ZOT, zona occludens toxin. 1. Advanced drug delivery reviews 35(2), 249-257 (1999).
6. Heinemann L. New ways of insulin delivery. International journal of clinical practice. Supplement doi:10.1111/j.1742-1241.2010.02577.x(170), 31-46 (2011).
7. Fonte P, Araujo F, Reis S, Sarmento B. Oral insulin delivery: how far are we? J Diabetes Sci Technol 7(2), 520-531 (2013).
8. Zijlstra E, Heinemann L, Plum-Morschel L. Oral insulin reloaded: a structured approach. J Diabetes Sci Technol 8(3), 458-465 (2014).
9. Zaykov A N, Mayer J P, Dimarchi R D. Pursuit of a perfect insulin. Nature reviews. Drug discovery 15(6), 425-439 (2016).
10. Wong C Y, Martinez J, Dass C R. Oral delivery of insulin for treatment of diabetes: status quo, challenges and opportunities. The Journal of pharmacy and pharmacology 68(9), 1093-1108 (2016).
11. Samson S L, Chan L. Gene therapy for diabetes: reinventing the islet Trends Endocrinol Metab 17(3), 92-100 (2006).
12. Alam T, Wai P, Held D, Vakili S T, Forsberg E, Sollinger H. Correction of Diabetic Hyperglycemia and Amelioration of Metabolic Anomalies by Minicircle DNA Mediated Glucose-Dependent Hepatic Insulin Production. PloS one 8(6), e67515 (2013).
13. Jariyapong P, Xing L, Van Houten N E et al. Chimeric hepatitis E virus-like particle as a carrier for oral-delivery. Vaccine 31(2), 417-424 (2013).
14. Chen C C, Xing L, Stark M et al. Chemically activatable viral capsid functionalized for cancer targeting. Nanomedicine (Loud) 11(4), 377-390 (2016).
15. Cheng R H, Xing L, Chen C C, Stark M C: WO/2015/179321 (2015).
16. Takamura S, Niikura M, Li T C et al. DNA vaccine-encapsulated virus-like particles derived from an orally transmissible virus stimulate mucosal and systemic immune responses by oral administration. Gene therapy 11(7), 628-635 (2004).
17. Cheng R H, Xing L: U.S. Pat. No. 8,906,863 (2014).
18. Stark M C, Baikoghli M A, Lahtinen T et al. Structural characterization of site-modified nanocapsid with monodispersed gold clusters. Scientific reports 7(1), 17048 (2017).
19. Li W, Chen X. Gold nanoparticles for photoacoustic imaging. Nanomedicine 10(2), 299-320 (2015).

1. A composition comprising:
(a) a modified capsid protein that comprises at least a portion of hepatitis E virus (HEV) open Reading Frame 2 (ORF2) protein and is able to form an HEV virus like particle (VLP); and
(b) an insulin protein or a nucleic acid encoding an insulin protein encapsulated within the HEV VLP formed by the modified capsid protein.

2. The composition of claim 1, wherein the modified capsid protein is less than full length of HEV ORF2 protein, comprises segment 452-606 of the HEV ORF 2 protein of SEQ ID NO:1, 2, 3, 4, 5, or 6, and comprises a heterologous polypeptide sequence inserted into the portion of HEV ORF2 protein within segment 483-490, 530-535, 554-561, 573-577, 582-593, or 601-603 of SEQ ID NO:1, 2, 3, 4, 5, or 6.

3. The composition of claim 2, wherein the heterologous polypeptide sequence is inserted immediately after residue Y485 of SEQ ID NO:1, 2, 3, 4, 5, or 6.

4. The composition of claim 2 [[or 3]], wherein the heterologous polypeptide is a RGD or cyclic RGD peptide.

5. The composition of claim 1, wherein the modified capsid protein is able to form an acid and proteolytically stable HEV VLP and has at least one residue Y485, T489, S533, N573, or T586 of SEQ ID NO:1, 2, 3, 4, 5, or 6 substituted with a cysteine or lysine, which is optionally chemically derivatized.

6. The composition of claim 4, wherein the cysteine or lysine is alkylated, acylated, arylated, succinylated, oxidized, or conjugated to a detectable label or liver cell targeting ligand.

7. The composition of claim 6, wherein the detectable label comprises a fluorophore, a superparamagnetic label, an MRI contrast agent, a positron emitting isotope, or a cluster of elements of group 3 through 18 having an atomic number greater than 20.

8. The composition of claim 7, wherein the detectable label comprises a gold nanocluster.

9. The composition of claim 6, wherein the liver cell targeting ligand is a RGD or cyclic RGD peptide.

10. The composition of claim 9, further comprising a pharmaceutically acceptable excipient.

11. The composition of claim 9, which is formulated for oral administration.

12. A method of targeted delivery of insulin comprising contacting a liver cell with the composition of claim 1.

13. The method of claim 12, wherein the liver cell is within a patient's body, and wherein the contacting step comprises administration of the composition of claim 1 to the patient.

14. The method of claim 12, wherein the administration is oral administration.

15. The method of claim 13, wherein the modified capsid protein comprises a cysteine or lysine conjugated to a gold nanocluster.

16. The method of claim 13, wherein the patient has been diagnosed with diabetes.


SEQUENCE LISTING (19 sequences)

SEQ ID NO: 1 - protein, 660 aa, Hepatitis E virus
MRPRPILLLLLMFLPMLPAPPPGQPSGRRRGRRSGGSGGGFWGDRADSQPFAIPYIHPTNPFAPDVTAAAGAGPRVRQPARPLGSAWRDQAQRPAAASRRRPTTAGAAPLTAVAPAHDTPPVPDVDSRGAILRRQYNLSTSPLTSSVATGTNLVLYAAPLSPLLPLQDGTNTHIMATEASNYAQYRVVRATIRYRPLVPNAVGGYAISISFWPQTTTTPTSVDMNSITSTDVRILVQPGIASEHVIPSERLHYRNQGWRSVETSGVAEEEATSGLVMLCIHGSLVNSYTNTPYTGALGLLDFALELEFRNLTPGNTNTRVSRYSSTARHRLRRGADGTAELTTTAATRFMKDLYFTSTNGVGEIGRGIALTLFNLADTLLGGLPTELISSAGGQLFYSRPVVSANGEPTVKLYTSVENAQQDKGIAIPHDIDLGESRVVIQDYDNQHEQDRPTPSPAPSRPFSVLRANDVLWLSLTAAEYDQSTYGSSTGPVYVSDSVTLVNVATGAQAVARSLDWTKVTLDGRPLSTTQQYSKTFFVLPLRGKLSFWEAGTTKAGYPYNYNTTASDQLLVENAAGHRVAISTYTTSLGAGPVSISAVAVLAPHSALALLEDTMDYPARAHTFDDFCPECRPLGLQGCAFQSTVAELQRLKMKVGKTREL

SEQ ID NO: 2 - protein, 660 aa, Hepatitis E virus
MRPRAVLLLFFVLLPMLPAPPAGQPSGRRRGRRSGGAGGGFWGDRVDSQPFALPYIHPTNPFAADVVSQSGAGARPRQPPRPLGSAWRDQSQRPSAAPRRRSAPAGAAPLTAISPAPDTAPVPDVDSRGAILRRQYNLSTSPLTSSVASGTNLVLYAAPLNPLLPLQDGTNTHIMATEASNYAQYRVVRATIRYRPLVPNAVGGYAISISFWPQTTTTPTSVDMNSITSTDVRILVQPGIASELVIPSERLHYRNQGWRSVETTGVAEEEATSGLVMLCIHGSPVNSYTNTPYTGALGLLDFALELEFRNLTPGNTNTRVSRYTSTARHRLRRGADGTAELTTTAATRFMKDLHFTGTNGVGEVGRGIALTLFNLADTLLGGLPTELISSAGGQLFYSRPVVSANGEPTVKLYTSVENAQQDKGITIPHDIDLGDSRVVIQDYDNQHEQDRPTPSPAPSRPFSVLRANDVLWLSLTAAEYDQTTYGSSTNPMYVSDTVTFVNVATGAQAVARSLDWSKVTLDGRPLTTIQQYSKTFYVLPLRGKLSFWEAGTTKAGYPYNYNTTASDQILIENAAGHRVAISTYTTSLGAGPTSISAVGVLAPHSALAVLEDTTDYPARAHTFDDFCPECRTLGLQGCAFQSTIAELQRLKMKVGKTRES

SEQ ID NO: 3 - protein, 660 aa, Hepatitis E virus
MRPRAVLLLFFVLLPMLPAPPAGQPSGRRRGRRSGGTGGGFWGDRVDSQPFALPYIHPTNPFASDIPTATGAGARPRQPARPLGSAWRDQSQRPAAPARRRSAPAGASPLTAVAPAPDTAPVPDVDSRGAILRRQYNLSTSPLTSTIATGTNLVLYAAPLSPLLPLQDGTNTHIIATEASNYAQYRVVRATIRYRPLVPNAVGGYAISISFWPQTTTTPTSVDMNSITSTDVRILVQPGIASELVIPSERLHYRNQGWRSVETSGVAEEEATSGLVMLCIHGSPVNSYTNTPYTGALGLLDFALELEFRNLTPGNTNTRVSRYSSSARHKLCRGPDGTAELTTTAATRFMKDLHFTGTNGVGEVGRGIALTLLNLADTLLGGLPTELISSAGGQLFYSRPVVSANGEPTVKLYTSVENAQQDKGIAIPHDIDLGESRVVIQDYDNQHEQDRPTPSPAPSRPFSVLRANDVLWLSLTAAEYDQTTYGSSTNPMYVSDTVTFVNVATGTQGVSRSLDWSKVTLDGRPLTTIQQYSKTFFVLPLRGKLSFWEAGTTKAGYPYNYNTTASDQILIENAPGHRVCISTYTTNLGSGPVSISAVGVLAPHSALAALEDTVDYPARAHTFDDFCPECRALGLQGCAFQSTVAELQRLKMKVGKTQEY

SEQ ID NO: 4 - protein, 659 aa, Hepatitis E virus
MRPRPLLLLFLLFLPMLPAPPTGQPSGRRRGRRSGGTGGGFWGDRVDSQPFAIPYIHPTNPFAPDVAAASGSGPRLRQPARPLGSTWRDQAQRPSAASRRRPATAGAAALTAVAPAHDTSPVPDVDSRGAILRRQYNLSTSPLTSSVASGTNLVLYAAPLNPPLPLQDGTNTHIMATEASNYAQYRVARATIRYRPLVPNAVGGYAISISFWPQTTTTPTSVDMNSITSTDVRILVQPGIASELVIPSERLHYRNQGWRSVETSGVAEEEATSGLVMLCIHGSPVNSYTNTPYTGALGLLDFALELEFRNLTTCNTNTRVSRYSSTARHSARGADGTAELTTTAATRFMKDLHFTGLNGVGEVGRGIALTLLNLADTLLGGLPTELISSAGGQLFYSRPVVSANGEPTVKLYTSVENAQQDKGVAIPHDIDLGDSRVVIQDYDNQHEQDRPTPSPAPSRPFSVLRANDVLWLSLTAAEYDQSTYGSSTGPVYISDSVTLVNVATGAQAVARSLDWSKVTLDGRPLPTVEQYSKTFFVLPLRGKLSFWEAGTTKAGYPYNYNTTASDQILIENAAGHRVAISTYTTRLGAGPVAISAAAVLAPRSALALLEDTFDYPGRAHTFDDFCPECRALGLQGCAFQSTVAELQRLKVKVGKTREL

SEQ ID NO: 5 - protein, 674 aa, Hepatitis E virus
  MOD_RES (368)..(368): Any amino acid
MNNMFLCFACGYATMRPRAILLLLVVLLPMLPAPPAGQSSGRRRGRRSGGAGSGFWGDRVDSQPFALPYIHPTNPFASDTIAATGTGARSRQSARPLGSAWRDQTQRPPAASRRRSTPTGASPLTAVAPAPDTRPVPDVDSRGAILRRQYNLSTSPLTSTIASGTNLVLYAAPLSPLLPLQDGTNTHIMATEASNYAQYRVVRATIRYRPLVPNAVGGYAISISFWPQTTTTPTSVDMNSITSTDVRIVVQPGLASELVIPSERLHYRNQGWRSVETSGVAEEEATSGLVMLCIHGSPVNSYTNTPYTGALGLLDFALELEFRNLTPGNTNTRVSRYSSTARHRLHRGADGTAELTTTAATRFMKDLXFTGSNGIGEVGRGIALTLFNLADTLLGGLPTELISSAGGQLFYSRPVVSANGEPTVKLYTSVENAQQDKGIAIPHDIDLGDSRVVIQDYDNQHEQDRPTPSPAPSRPFSVLRVNDVLWLTMTAAEYDQTTYGTSTDPVYVSDTVTFVNVATGAQGVARSLDWSKVTLDGRPLTTIQRHSKNYFVLPLRGKLSFWEAGTTKAGYPYNYNTTASDQILIENAAGHRVCISTYTTSLGSGPVSVSGVGVLAPHAALAVLEDTVDYPARAHTFDDFCPECRTLGLQGCAFQSTVAELQRLKMRVGKTREF

SEQ ID NO: 6 - protein, 660 aa, Hepatitis E virus
MRPRAVLLLFLMLLPMLPAPPAGQPSGRRRGRRSGGSGGGFWGDRVDSQPFALPYIHPTNPFASDVSTSAGAGARARQAARPLGSAWRDQSQRPSASARRRPTPAGASPLTAVAPAPDTTPVPDVDSRGAILRRQYNLSTSPLTSTVASGTNLVLYAAPLGPLLPLQDGTNTHIMATEASNYAQYRVIRATIRYRPLVPNAVGGYAISISFWPQTTTTPTSVDMNSITSTDVRILVQPGLASELIIPSERLHYRNQGWRSVETSGVAEEEATSGLVMLCIHGSPVNSYTNTPYTGALGLLDFALELEFRNLTPGNTNTRVSRYTSTARHRLRRGPDGTAELTTTAATRFMKDLYFTGSNGLGEVGRGIALTLFNLADTLLGGLPTELISSAGGQLFYSRPVVSANGEPTVKLYTSVENAQQDKGIAIPHEIDLGDSRVTIQDYDNQHEQDRPTPSPAPSRPFSVLRVNDVLWLTLTAAEYDQTTYGSTTNPMYVSDTVTFVNVATGAQGVARALDWSKVTFDGRPLTTVQQYGKSFFVLPLRGKLSFWEAGTVKAGYPYNYNTTASDQILVENAPGHRVCISTYTTNLGSGPVSISAVGVLAPHAATAALEDTADSPARAHTFDDFCPECRILGLQGCAYQSTAAELQRLKMKVGKSREF

SEQ ID NO: 7 - protein, 497 aa, Hepatitis E virus
AVAPAHDTPPVPDVDSRGAILRRQYNLSTSPLTSSVATGTNLVLYAAPLSPLLPLQDGTNTHIMATEASNYAQYRVVRATIRYRPLVPNAVGGYAISISFWPQTTTTPTSVDMNSITSTDVRILVQPGIASEHVIPSERLHYRNQGWRSVETSGVAEEEATSGLVMLCIHGSLVNSYTNTPYTGALGLLDFALELEFRNLTPGNTNTRVSRYSSTARHRLRRGADGTAELTTTAATRFMKDLYFTSTNGVGEIGRGIALTLFNLADTLLGGLPTELISSAGGQLFYSRPVVSANGEPTVKLYTSVENAQQDKGIAIPHDIDLGESRVVIQDYDNQHEQDRPTPSPAPSRPFSVLRANDVLWLSLTAAEYDQSTYGSSTGPVYVSDSVTLVNVATGAQAVARSLDWTKVTLDGRPLSTTQQYSKTFFVLPLRGKLSFWEAGTTKAGYPYNYNTTASDQLLVENAAGHRVAISTYTTSLGAGPVSISAVAVLAPHSALA

SEQ ID NO: 8 - protein, 497 aa, Hepatitis E virus
AISPAPDTAPVPDVDSRGAILRRQYNLSTSPLTSSVASGTNLVLYAAPLNPLLPLQDGTNTHIMATEASNYAQYRVVRATIRYRPLVPNAVGGYAISISFWPQTTTTPTSVDMNSITSTDVRILVQPGIASELVIPSERLHYRNQGWRSVETTGVAEEEATSGLVMLCIHGSPVNSYTNTPYTGALGLLDFALELEFRNLTPGNTNTRVSRYTSTARHRLRRGADGTAELTTTAATRFMKDLHFTGTNGVGEVGRGIALTLFNLADTLLGGLPTELISSAGGQLFYSRPVVSANGEPTVKLYTSVENAQQDKGITIPHDIDLGDSRVVIQDYDNQHEQDRPTPSPAPSRPFSVLRANDVLWLSLTAAEYDQTTYGSSTNPMYVSDTVTFVNVATGAQAVARSLDWSKVTLDGRPLTTIQQYSKTFYVLPLRGKLSFWEAGTTKAGYPYNYNTTASDQILIENAAGHRVAISTYTTSLGAGPTSISAVGVLAPHSALA

SEQ ID NO: 9 - protein, 497 aa, Hepatitis E virus
AVAPAPDTAPVPDVDSRGAILRRQYNLSTSPLTSTIATGTNLVLYAAPLSPLLPLQDGTNTHIIATEASNYAQYRVVRATIRYRPLVPNAVGGYAISISFWPQTTTTPTSVDMNSITSTDVRILVQPGIASELVIPSERLHYRNQGWRSVETSGVAEEEATSGLVMLCIHGSPVNSYTNTPYTGALGLLDFALELEFRNLTPGNTNTRVSRYSSSARHKLCRGPDGTAELTTTAATRFMKDLHFTGTNGVGEVGRGIALTLLNLADTLLGGLPTELISSAGGQLFYSRPVVSANGEPTVKLYTSVENAQQDKGIAIPHDIDLGESRVVIQDYDNQHEQDRPTPSPAPSRPFSVLRANDVLWLSLTAAEYDQTTYGSSTNPMYVSDTVTFVNVATGTQGVSRSLDWSKVTLDGRPLTTIQQYSKTFFVLPLRGKLSFWEAGTTKAGYPYNYNTTASDQILIENAPGHRVCISTYTTNLGSGPVSISAVGVLAPHSALA

SEQ ID NO: 10 - protein, 497 aa, Hepatitis E virus
AVAPAHDTSPVPDVDSRGAILRRQYNLSTSPLTSSVASGTNLVLYAAPLNPPLPLQDGTNTHIMATEASNYAQYRVARATIRYRPLVPNAVGGYAISISFWPQTTTTPTSVDMNSITSTDVRILVQPGIASELVIPSERLHYRNQGWRSVETSGVAEEEATSGLVMLCIHGSPVNSYTNTPYTGALGLLDFALELEFRNLTTCNTNTRVSRYSSTARHSARGADGTAELTTTAATRFMKDLHFTGLNGVGEVGRGIALTLLNLADTLLGGLPTELISSAGGQLFYSRPVVSANGEPTVKLYTSVENAQQDKGVAIPHDIDLGDSRVVIQDYDNQHEQDRPTPSPAPSRPFSVLRANDVLWLSLTAAEYDQSTYGSSTGPVYISDSVTLVNVATGAQAVARSLDWSKVTLDGRPLPTVEQYSKTFFVLPLRGKLSFWEAGTTKAGYPYNYNTTASDQILIENAAGHRVAISTYTTRLGAGPVAISAAAVLAPRSALAL

SEQ ID NO: 11 - protein, 497 aa, Hepatitis E virus
  MOD_RES (257)..(257): Any amino acid
SRRRSTPTGASPLTAVAPAPDTRPVPDVDSRGAILRRQYNLSTSPLTSTIASGTNLVLYAAPLSPLLPLQDGTNTHIMATEASNYAQYRVVRATIRYRPLVPNAVGGYAISISFWPQTTTTPTSVDMNSITSTDVRIVVQPGLASELVIPSERLHYRNQGWRSVETSGVAEEEATSGLVMLCIHGSPVNSYTNTPYTGALGLLDFALELEFRNLTPGNTNTRVSRYSSTARHRLHRGADGTAELTTTAATRFMKDLXFTGSNGIGEVGRGIALTLFNLADTLLGGLPTELISSAGGQLFYSRPVVSANGEPTVKLYTSVENAQQDKGIAIPHDIDLGDSRVVIQDYDNQHEQDRPTPSPAPSRPFSVLRVNDVLWLTMTAAEYDQTTYGTSTDPVYVSDTVTFVNVATGAQGVARSLDWSKVTLDGRPLTTIQRHSKNYFVLPLRGKLSFWEAGTTKAGYPYNYNTTASDQILIENAAGHRVCISTYTTSLGSGPVS

SEQ ID NO: 12 - protein, 497 aa, Hepatitis E virus
AVAPAPDTTPVPDVDSRGAILRRQYNLSTSPLTSTVASGTNLVLYAAPLGPLLPLQDGTNTHIMATEASNYAQYRVIRATIRYRPLVPNAVGGYAISISFWPQTTTTPTSVDMNSITSTDVRILVQPGLASELIIPSERLHYRNQGWRSVETSGVAEEEATSGLVMLCIHGSPVNSYTNTPYTGALGLLDFALELEFRNLTPGNTNTRVSRYTSTARHRLRRGPDGTAELTTTAATRFMKDLYFTGSNGLGEVGRGIALTLFNLADTLLGGLPTELISSAGGQLFYSRPVVSANGEPTVKLYTSVENAQQDKGIAIPHEIDLGDSRVTIQDYDNQHEQDRPTPSPAPSRPFSVLRVNDVLWLTLTAAEYDQTTYGSTTNPMYVSDTVTFVNVATGAQGVARALDWSKVTFDGRPLTTVQQYGKSFFVLPLRGKLSFWEAGTVKAGYPYNYNTTASDQILVENAPGHRVCISTYTTNLGSGPVSISAVGVLAPHAATA

SEQ ID NO: 13 - protein, 7 aa, Artificial Sequence
  Description of Artificial Sequence Synthetic peptide
TTPRDAY

SEQ ID NO: 14 - protein, 7 aa, Artificial Sequence
  Description of Artificial Sequence Synthetic peptide
FQHPSFI

SEQ ID NO: 15 - protein, 12 aa, Artificial Sequence
  Description of Artificial Sequence Synthetic peptide
SFSIIHTPILPL

SEQ ID NO: 16 - protein, 12 aa, Artificial Sequence
  Description of Artificial Sequence Synthetic peptide
RGWCRPLPKGEG

SEQ ID NO: 17 - protein, 12 aa, Artificial Sequence
  Description of Artificial Sequence Synthetic peptide
AGKGTPSLETTP

SEQ ID NO: 18 - protein, 12 aa, Artificial Sequence
  Description of Artificial Sequence Synthetic peptide
KSLSRHDHIHHH

SEQ ID NO: 19 - protein, 7 aa, Artificial Sequence
  Description of Artificial Sequence Synthetic peptide
AWYPLPP